(19) United States
(12) Patent Application Publication
(10) Pub. No.: US 2014/0235984 A1
(43) Pub. Date: Aug. 21, 2014

(54) SYSTEM FOR A BLOOD ANALYTE SENSOR

(71) Applicant: Edwards Lifesciences Corporation, Irvine, CA (US)

(72) Inventors: Jennifer L. Wilbur, Newport Beach, CA (US); Michael Higgins, Huntington Beach, CA (US); Paul S. Van Wieren, Costa Mesa, CA (US); Alexander H. Siemons, Yorba Linda, CA (US); Chris J. Okos, Huntington Beach, CA (US)

(73) Assignee: Edwards Lifesciences Corporation, Irvine, CA (US)

(21) Appl. No.: 14/168,932

(22) Filed: Jan. 30, 2014

Related U.S. Application Data
(60) Provisional application No. 61/760,013, filed on Feb. 1, 2013.

(51) Int. Cl. A61B 5/15; A61B 5/157; A61B 5/155

(52) U.S. Cl. CPC A61B 5/150992; A61B 5/155; A61B 5/157
USPC 600/365

(57) ABSTRACT

The present invention provides a blood parameter sensing system that may include a sensor, a blood access system, a flow controller and a monitor system. The blood access system may include a blood draw member having an internal configuration adapted to avoid clotting without the use of anticoagulants and under a range of flow profiles, including small volume blood draws. The flow controller may be configured to adapt to a range of access devices by using feedback from the sensor and monitor to customize flow profiles. The monitor system is configured to reduce parameter measurement error by modeling sensitivity change during system startup or reconnect.

### CROSS-REFERENCE TO RELATED APPLICATIONS

This application claims the benefit of U.S. Provisional Application No. 61/760,013, filed Feb. 1, 2013, which is incorporated by reference herein.

### FIELD OF THE INVENTION

This invention relates to intravascular blood analyte sensor systems and, in particular, a flow control system for blood parameter sensor systems that controls small flow volumes.

### BACKGROUND OF THE INVENTION

Analyte testing in the home is fairly common and involves the use of finger stick glucometers that return blood glucose levels on an intermittent basis throughout a day. For patients in a hospital setting, however, these intermittent tests are not frequent enough to capture a patient's (usually) more dynamically changing condition. Patients in critical care settings can experience especially high fluctuations in blood analytes such as glucose. Tracking such changes is better accomplished by more frequent sampling and reporting of analyte levels. To this end, companies have recently been developing continuous glucose monitoring systems for the hospital.

In continuous analyte monitoring the same sensor is repeatedly called upon over a series of hours or days to report sensed analyte parameters. During this time, it's advantageous to ensure that the sensor is correctly calibrated by use of a calibration fluid with a known concentration of the analyte of interest, such as glucose. For example, a sensor system employed by Edwards Lifesciences, Corporation (Irvine, Calif.) uses a glucose-oxidase sensor mounted in a catheter tube to sense patient glucose levels continuously for up to 72 hours. This analyte sensor is cycled in alteration continuously, during this three-day period, through blood sample sensing cycles and calibration cycles in the present of calibrant to ensure high accuracy.

During the calibration cycle, calibrant fluid is flowed through tubing by a flow control device over the sensor and the sensor reading is adjusted to match the known concentration of calibrant fluid. The calibrant fluid originates from a bag filled with standard 0.9% NaCI saline, dextrose and may or may not contain an anti-coagulant, such as heparin. This calibrant fluid is created by taking a standard saline bag of known volume (or weight) and mixing in a known volume (or weight) of dextrose.

During sensing cycles, a vacuum pressure is generated in the catheter tube which draws blood from the patient's vasculature through the distal tip of the catheter. In the Edwards sensor system, the catheter (e.g., a JELCO 20Ga×1.25 inch) holds the glucose sensor at its distal tip and therefore only requires approximately a small (e.g., 40-200 μL) draw for enough blood to bathe the sensor and allow sensing of the glucose concentration. Typically, this catheter is inserted over a needle.

In its current form, the Edwards sensor system uses a dedicated catheter. However, it may be advantageous to insert the sensor into pre-existing catheter in use with a patient. Many patients in the hospital may already have several lines or multi-lumen catheters in place for drawing blood, sensing hemodynamic parameters, injecting drugs, etc. Insertion of the analyte sensor into one of these existing catheters would eliminate the need for a separate, dedicated catheter.

### SUMMARY OF THE INVENTION

The present invention overcomes the problems of the prior art by providing a blood parameter sensing system that includes a sensor, a blood access system, a flow controller and a monitor system. Various embodiments of the blood access system can include a blood draw member having an internal configuration adapted to avoid clotting without the use of anticoagulants and under a range of flow profiles. The flow controller may be configured to adapt to a range of access devices by using feedback from the sensor and monitor to customize flow profiles. The monitor system is configured to reduce parameter measurement error by modeling sensitivity change during system startup or reconnect.

The present invention in one embodiment includes a blood access system for use with a system for sampling blood from a patient's vasculature and directing the blood to a blood parameter sensor. The blood access system includes a blood draw member with a proximal end and a distal end. The blood draw member defines at least one lumen extending from the proximal end to the distal end. The distal end of the blood draw member resides within the patient's vasculature for an extended period of time. At its opposite, proximal end the blood draw member is connectable in fluid communication with the blood parameter sensor. An inside surface of the blood draw member that defines the lumen is smooth, continuous and void-free so as to avoid clotting. An inside diameter of the lumen of the blood draw member is less than 0.025 inches so as to avoid clotting during flush and draw pressures of up to 10 psi. Smaller diameters are possible, although they would require higher pumping pressures. In some embodiments, the blood draw member comprises a coiled portion adjacent to said proximal end, such that said coil portion increases the overall length of the blood draw member.

In another embodiment, the inside diameter of the blood draw member is within a range of 0.008 inches to 0.015 inches. An outside diameter of the blood draw member is within a range of 0.015 inches to 0.025 inches. Also, a difference between the inside and outside diameters may be at least 0.005 inches, or even 0.015 inches or greater for improved buckling stiffness. A ratio of the outside to inside diameters may be 2.5 times or greater, again, improving buckling stiffness. In another embodiment, buckling or kinking stiffness may also be increased through use of some axial structure on the inside surface of the blood draw member. Generally, in these embodiments it is desirable that the buckling strength is sufficient to withstand direct insertion into the patient's vasculature.

Embodiments may also include different materials for the blood draw member such as polyurethane, polyimide and/or nylon.

In another embodiment, the inside diameter of the blood draw member may increase as its lumen extends from the distal end to the proximal end. The inside diameter, alternatively, may also be constant between the two ends. In another aspect, the blood access member may define a plurality of lumens.

Preferably, to avoid clotting, the inside surface is smooth, continuous and void-free. Further preferably, the blood draw member is configured to be clot-free up to a maximum draw rate of 200 mL/hr.

In another embodiment, the blood draw member is configured for connection at its proximal end into fluid communication with a monitoring line from which it receives a fluid solution. Preferably, the inside diameter of the lumen of the blood draw member is at least 0.002 inches less than an inside diameter of a lumen of the monitoring line. Or, the inside diameter of the blood draw member is at least 25% less than an inside diameter of a lumen of the monitoring line. Or, the inside diameter of the lumen of the blood draw member is 0.010 inches and an inside diameter of a lumen of the monitoring line is 0.012 inches.

In another embodiment, the blood access system includes an adapter having a distal end configured for attachment to the blood draw member and a proximal end configured for attachment to a sensor casing. The adapter may define a lumen having a proximal end with an inside diameter matched to a lumen of the sensor casing and a distal end with an inside diameter matched to the lumen of the blood draw member. A surface defining the lumen is preferably smooth, continuous and void-free so as to avoid clotting during flush and draw pressures of up to 10 psi. The adapter lumen may also have some taper, such as with an inside diameter of 0.020 inches at the proximal end and 0.010 inches at the distal end.

In another embodiment, the sensor casing is included in the blood access system. The sensor casing may be configured to support a blood parameter sensor and includes a proximal end configured for attachment to a monitoring line and a distal end configured for attachment to the proximal end of the adapter.

In another embodiment, the blood access system includes the monitoring line. An inside diameter of the lumen of the blood draw member is at least 0.002 inches less than an inside diameter of a lumen of the monitoring line. Or, the inside diameter of the blood draw member is at least 25% less than an inside diameter of a lumen of the monitoring line. Or, the inside diameter of the lumen of the blood draw member is 0.010 inches and an inside diameter of a lumen of the monitoring line is 0.012 inches. Preferably, the monitoring line is at least 8 feet long.

In another embodiment, the blood access system includes a guide wire configured to extend through the lumen of the blood draw member. And, an introducer may be included, the introducer configured to fit over the guide wire and to be withdrawn from the guide wire after insertion into the patient's vasculature.

In another embodiment, the blood access system includes a tear-away introducer configured to insert into the patient's vasculature. The tear-away introducer defines a lumen configured to receive the blood draw member.

In another embodiment, the present invention includes a blood parameter sensor system for sensing a parameter in blood accessed by an access device. Included in the blood parameter sensor system are a blood parameter sensor, flow control system and monitor. The blood parameter sensor is coupled to the access device. The flow control system is configured to draw blood through the access device to the blood parameter sensor and flush the sensor with a calibrant. The monitor is connected in communication with the blood parameter sensor. The monitor is configured to receive continuous signal such that a blood signal will be identified when the sensor is in blood and receive similarly a calibration signal when the sensor is in the calibrant. The blood parameter is determined from the blood signal by the monitor using the calibration signal.

Preferably, a first delay between the receipt of the blood signal and the calibration signal is shorter than a second delay between receipt of a previous calibration signal and the blood signal. For example, the first delay may be at least 1.5 minutes shorter than the second delay, or even at least 2.5 minutes shorter. In another aspect, the blood signal may be received at an end of the blood draw and the calibration signal received within a first ⅔ of a calibrant flush cycle.

In another embodiment, the present invention includes a blood parameter sensor system for sensing a parameter in blood. The blood parameter sensor system includes a blood parameter sensor, a flow control system and a monitor. The flow control system is configured to expose the blood parameter sensor to a calibrant. The monitor is connected in communication with the blood parameter sensor. It is configured to receive a calibration signal and a blood signal, wherein a delay occurs between the calibration signal and the blood signal. Also, the monitor is configured to modify the calibration signal to account for the delay and use the modified calibration signal to determine the parameter from the blood signal.

A second signal may be communicated to the monitor wherein the monitor uses the second calibration signal to modify the calibration signal. For instance, the calibration signal may be modified using a statistical method, such as an extrapolative linear regression method.

In yet another embodiment, the present invention includes a blood parameter sensor, a flow control system and a monitor. The flow control system is configured to flow blood past the sensor. The monitor is connected in communication with the blood parameter sensor and is configured to receive a blood signal from the blood parameter sensor. It determines a blood parameter from the blood signal. And, the flow control system is configured to continuously flow blood past the sensor while the blood parameter sensor generates the blood signal.

In one aspect, the blood signal includes a waveform and the flow control system is configured to determine a plateau threshold from the waveform. The waveform may include a plurality of blood signal readings. Blood parameters associated with a portion of the blood signal exceeding the plateau threshold are reported by the monitor. The monitor may be further configured to use the plateau threshold to determine if the blood is homogenous.

In another embodiment, the monitor may be further configured to determine when the waveform does not meet the plateau threshold, such as within a maximum delay period, as an indicator of non-homogeneity.

The flow control system may be further configured to continuously flow blood until a clinical event occurs, such as a draw of a clinically relevant volume of blood, at which point the blood is flushed out of an access device.

In another embodiment, the present invention includes a blood parameter sensor system for sensing a parameter in blood accessed by an access device. The blood parameter sensor system includes a blood parameter sensor, a monitor and a flow control system. The blood parameter sensor is supported by an access device. Connected in communication with the blood parameter sensor is the monitor. The monitor is configured to receive a blood signal from the blood parameter sensor and determine a blood parameter waveform from the blood signal. Also, the monitor is configured to determine a threshold from the blood parameter waveform. The flow control system is connected in communication with the monitor. The flow control system is connected in communication with the monitor and is configured to draw blood into the access device to the sensor. And the flow control system is configured to receive the threshold from the monitor and determine a blood draw volume from the threshold.

Preferably, the blood draw volume is the volume of blood drawn past the sensor when the threshold is reached by the blood parameter waveform. The threshold may be a plateau threshold defining a relatively flat portion of the waveform.

In another aspect, the flow control system is configured to adapt a flow profile using the blood volume. For example, the flow profile may be adapted by increasing a draw rate over a fixed draw period to reach the blood draw volume. Alternatively, the draw rate may be fixed and the draw period modified to reach the blood draw volume.

In another aspect, the flow control system is configured to determine a plurality of blood draw volumes and adapt the flow profile using the plurality of blood draw volumes. Each of the plurality of blood draw volumes may be associated with an initialization flow profile, each having a different flow rate.

In addition, the flow control system may adapt the flow profile using a lookup table stored on a database.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

FIG. 1 is a perspective view of an analyte sensing system of one embodiment of the present invention;

FIG. 2 is a cross-sectional view of components, including a blood draw line, of a flow control system of the analyte sensing system shown in FIG. 1;

FIG. 3 is an enlarged view of an adapter of the components shown in FIG. 2;

FIG. 4 is a perspective view of the components, including a blood draw line, shown in FIG. 2;

FIG. 5 is a schematic of a rotary pinch valve of a flow control system of an embodiment of the present invention;

FIGS. 6a and 6b are front views of a rotary pinch valve of a flow control system of an embodiment of the present invention;

FIGS. 7a and 7b are front views of a rotary pinch valve of a flow control system of an embodiment of the present invention;

FIG. 8 is a perspective view of an adapter of the components shown in FIG. 2;

FIG. 9 is a perspective view of an adapter of another embodiment of the present invention;

FIG. 10 is a cross-sectional view of a catheter for combination with a blood draw line 28 of another embodiment of the present invention;

FIG. 11 is a perspective view of a blood draw line and dedicated catheter of another embodiment of the present invention;

FIG. 12 is a cross-sectional view of a flow control system with a hub for connection to a blood draw line of another embodiment of the present invention;

FIG. 13 is a perspective view of a tear away introducer for use with a blood draw line of another embodiment of the present invention;

FIG. 14 is a graphical depiction of a flow profile of an embodiment of the present invention;

FIG. 15 is a perspective view of an analyte sensor assembly according to another embodiment of the present invention;

FIG. 16 is an exploded perspective view of the sensor assembly of FIG. 15;

FIGS. 17a and 17b are perspective views of the first electronic housing subassembly of the sensor assembly of FIG. 15;

FIG. 18 is a cross-sectional view of the sensor and the first electronic housing subassembly of the sensor assembly of FIG. 15;

FIG. 19 is a cross-sectional view of the second electronic housing subassembly of the sensor assembly of FIG. 15;

FIG. 20a is a perspective view of an integrated monitor according to an embodiment of the present invention;

FIG. 20b is perspective view of a cassette cartridge for coupling the flush line with the flow controller according to one embodiment of the present invention;

FIG. 20c is perspective view of the integrated monitor of FIG. 20a illustrating connection of the cassette cartridge of FIG. 20b;

FIGS. 21a and 21b are exploded perspective views of the cassette cartridge of FIG. 20b;

FIG. 22 is a three-dimensional graph of modeled results for determining a calibration flush pressure as a function of monitoring line and sampling line diameter of varied embodiments of the present invention;

FIG. 23 is a three-dimensional graph of modeled results of blood visibility in a flush line as a function of monitoring line and sampling line diameter of varied embodiments of the present invention;

FIG. 24 is a three-dimensional graph of modeled results of pump infusion (flush) pressures as a function of hold time (for sensing a calibration value) and sampling line diameter of varied embodiments of the present invention;

FIG. 25 is a three-dimensional graph of modeled results of pump sample draw pressures as a function of hold time (for sensing a calibration value) and sampling line diameter of varied embodiments of the present invention;

FIG. 26 is a line graph of another embodiment of the present invention showing sensor current over time through alternating calibration and blood sampling cycles;

FIG. 27 is an enlarged view of a portion of the line graph of FIG. 26 showing, in another embodiment, a linear regression of the calibration sensitivity;

FIG. 28 is a line graph of another embodiment of the present invention showing post-blood sampling calibration to compress drift error;

FIGS. 29A & 29B are error plots showing reduced error on run-in using the embodiments of FIGS. 26-28;

FIGS. 30A & 30B are error plots showing reduced error on disconnect-reconnecting using the embodiments of FIGS. 26-28;

FIG. 31 is a schematic of multiple initializations of another embodiment of the present invention proceeding application of a nominal profile;

FIG. 32 is a line graph of another embodiment of the present invention showing a threshold plateau indicating immersion of a sensor in a sample;

FIG. 33 is a line graph of another embodiment of the present invention showing threshold plateaus for different blood access device configurations;

FIG. 34 is a line graph of another embodiment of the present invention including continuous blood draw and sampling;

FIG. 35 is a line graph of another embodiment of the present invention with continuous blood draw and sampling over a longer cycle;

FIG. 36 is a line graph of another embodiment of the present invention where blood draws alternate between draw and flush during continuous blood parameter sensor sampling;

FIG. 37 is a flowchart of a reverse-order calibration of an embodiment of the present invention;

FIG. 38 is a flowchart of a sensor drift extrapolation of an embodiment of the present invention;

FIG. 39 is a flowchart of a high-resolution, continuous sensing embodiment of the present invention;

FIG. 40 is a flowchart of an initialization routine of another embodiment of the present invention; and

FIG. 41 is a flowchart of a blood parameter sensing computer system of another embodiment of the present invention.

### DETAILED DESCRIPTION OF THE INVENTION

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

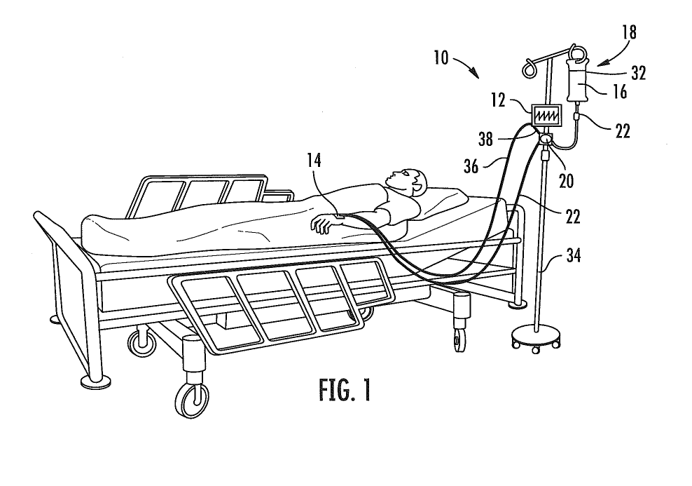

Embodiments of the present invention include a blood glucose sensing system 10 that includes a monitor 12, a sensor assembly 14, a calibration solution source 16 and a flow control system 18, as shown in FIG. 1. Notably, the present invention could also be employed with other analyte or blood parameter sensing systems that require drawing of blood or fluid samples from a patient. Blood, as used herein, should be construed broadly to include any body fluid with a tendency to occlude lumens of various body-access devices during sampling. The flow control system 18 includes a flow controller 20, a flush line 22, a sensor casing 24, an adapter 26, a blood draw line 28 and a catheter 30, as shown in FIGS. 1, 2, 4 and 5. Generally, the flow control system 18 of one embodiment of the present invention is configured to mediate flow of small volumes of the calibration solution 16 over the sensor assembly 14 and withdraw small volumes of samples of the blood from the patient for testing by the sensor assembly.

The flow control system 18 in another embodiment is able to support the flush and draw pressures and volumes, and the high number of sampling cycles over a long multi-day indwell, needed for continuous analyte (glucose) monitoring, while avoiding the formation of thrombi that occur in conventional catheters by providing a small-diameter, smooth and relatively void free surface defining a lumen extending up to the sensor assembly 14. In another embodiment, the blood draw line 28 of the flow control system 18 may be employed with a range of existing catheter 30 configurations by having the blood draw line 28 sized and configured for insertion into a lumen of an existing catheter. In still other embodiments of the present invention, thrombus formation is inhibited by balancing the structure of various components of the flow control system 18 and operation of the flush and draw cycles by the flow controller 20.

The monitor 12 is connected in communication with the sensor assembly 14 through a monitoring line comprising communication lines or wires 36 and to the flow control system 18 through the monitoring line, as shown in FIG. 1. The monitoring lines 36, 38 could also represent wireless data communication such as cellular, WIFI, RF, infrared or blue-tooth communication. Regardless, the monitor 12 includes some combination of hardware, software and/or firmware configured to record and display data reported by the sensor assembly 14. For example, the monitor may include processing and electronic storage for tracking and reporting blood glucose levels. In addition, the monitor 12 may be configured for automated control of various operations of other aspects of the sensing system 10. For example, the monitor 12 may be configured to operate the flow control system 18 to flush the sensor assembly 14 with calibration solution from source 16 and/or to draw samples of blood for testing by the sensor assembly. Also, the monitor 12 can be configured to calibrate the sensor assembly 14 based on the flush cycle. In some embodiments, the potentiostat used to communicate with the sensor can be part of the monitor or it can be located in the monitoring line. In the configuration in which the potentiostat is in the monitoring line, it may be located adjacent to the sensor to reduce signal noise.

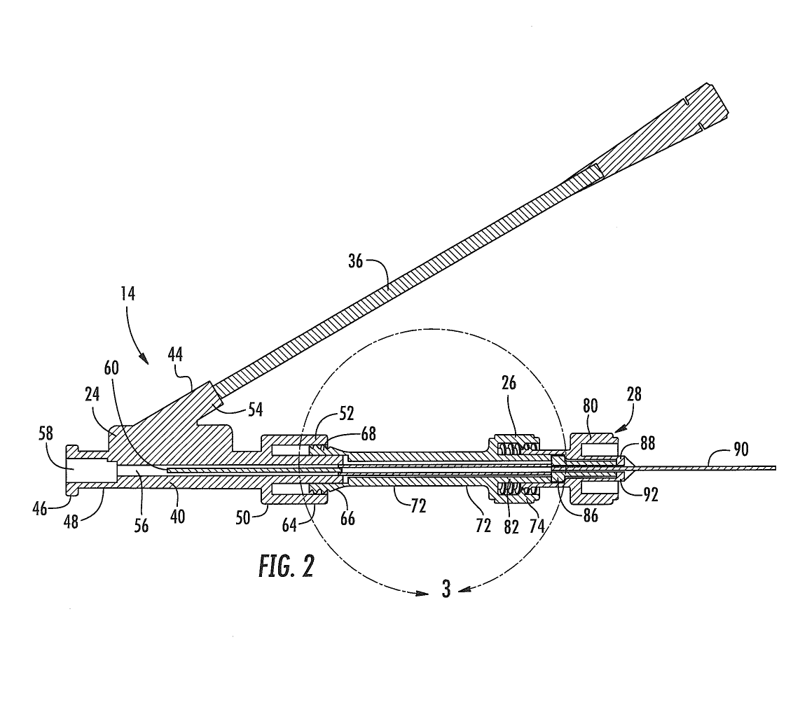

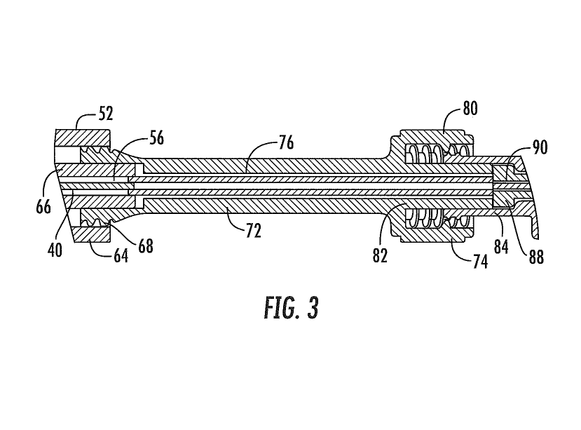

Referring to FIGS. 2 and 3, the sensor assembly 14 includes a wire electrode sensor 40 that includes, for example, a glucose-oxidase coated platinum wire covered by a membrane that selectively allows permeation of glucose. The glucose-oxidase responds to the glucose by generating hydrogen peroxide which, in turn, generates an electrical signal in the platinum wire. The platinum wire is connected to an electronic connection board held in a housing 44 of the sensor assembly 14. The electronic connection board may include some processing component and/or just communicate the signal up through the monitoring line (communication wires 36) attached thereto for further processing by the monitor 12. The electronic connection board is discussed later below. The sensor assembly 14 may also include counter and/or reference wire electrodes bundled with the working electrode. Regardless, in the illustrated embodiment, the wire electrode sensor 40 is adapted to extend through and into the sensor casing 24 so as to be within the flow path of the blood sample, as will be described in more detail herein below.

It should be noted that, although particularly advantageous for sensors 40 directly within the flow path of the blood sample, the particular configuration of the sensor assembly 14 that puts it within the flow of the blood and/or calibrant path may vary and still be within the scope of the present invention. For example, the sensor 40 could be a microfluidics sensor that is adjacent to, and routed off of, a portion of the flow control system 18 within the reach of a blood volume draw. Also, the sensor 40 could be an optical or vibrational sensor that senses blood parameters without contact with the blood sample, such as through a vibrationally or optically transparent adjacent portion of the flow control system.

Returning to FIG. 1, the calibrant solution source 16 is supplied, in one embodiment, from a bag 32 mounted on a pole 34. The calibrant solution supply is preferably off-the-shelf and/or not inconvenient to employ in a hospital setting and is also beneficial to the patient and includes attributes that help with function of the sensing system 10. For example, the solution in the bag may be a Plasmalyte or conventional saline with selected amounts of buffers and anti-thrombogenic compounds, such as heparin, that help with flushing the sensor assembly 14 to keep it clear of clots and thrombosis. The solution in the bag 32 may also include various nutrients to keep fluid and nutrition at appropriate levels for the patient. Although the illustrated embodiment employs a fluid bag 32, it should be noted that the calibrant solution source 16 could include several sources, including several sources at one time, and have varying compositions. For example, a pressurized canister or a reservoir may be employed.

As shown in FIG. 1, the flush line 22 of an embodiment of the flow control system 18 extends from the calibrant solution source 16 through the flow controller 20 and attaches to the rest of the flow control system 18 (sensor casing 24, adapter 26 and blood draw line 28 within catheter 30) closer to the sensor assembly 14. Preferably, the monitoring line is an 8 foot length of tubing with a 0.0625 inch internal diameter. The monitoring may comprise different materials along its length. For example, PVC tubing may be used for most of the line. However, the section in contact with the rotor valve of the flow controller (discussed later below) is generally comprised of a more pliable material, such as silicone. In some embodiments, a cassette assembly is as part of the monitoring line between the bag and flow controller and PVC flush line is provided between the flow controller and the sensor. The cassette includes an IV spike, optional drip chamber, IV tubing, silicone tubing and an extension PVC tubing/luer.

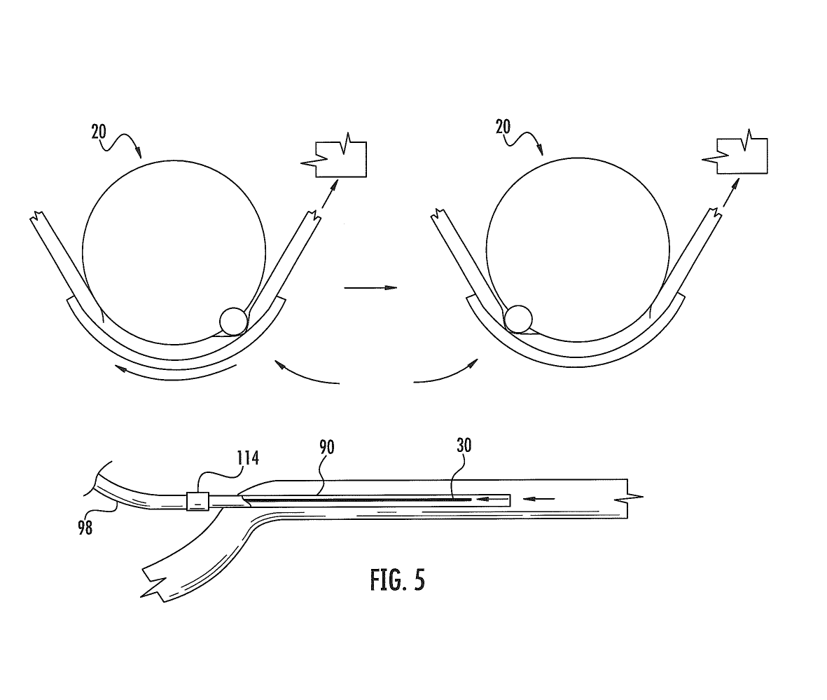

The flow controller 20 in one embodiment of the present invention includes some type of hardware, software, firmware or combination thereof that electromechanically controls one or more valves, or other mechanical flow control devices, to selectively allow or stop flow through the flush line 22. In the illustrated embodiment of FIG. 5, the mechanical aspect of the flow controller 20 includes a rotary pinch valve through which extends the flush line 22. This rotary pinch valve pinches the fluid line to stop flow and, by sliding along a short length of the fluid line, can advance or retract the calibrant solution or retract the calibrant solution supply in a column extending down to the end of the catheter 30. Different numbers of roller heads may be used, such as two or four heads, the latter aiding with higher draw volumes and/or efficient movement of the valve.

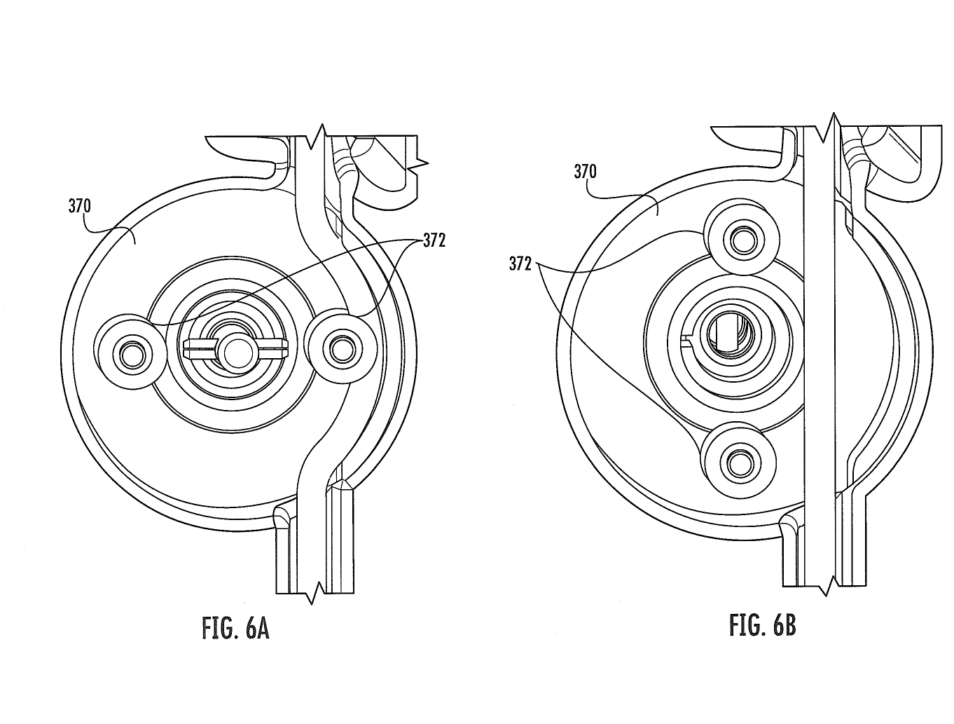

For example, FIGS. 6a and 6b disclose an alternative embodiment of the flow controller that employs a dual roller flow controller 370. As illustrated the dual roller flow controller comprises a set of two rollers 372 for contacting the IV line to effect flow control. The two roller system allows the system to more quickly and easily transition between draw, flush, and open valve states, such as for example to implement continuous flow in the IV line. The use of multiple rollers is also and aid for higher draw volumes. FIG. 6a illustrates a closed valve state, and FIG. 6b illustrates an open valve state for the flow controller.

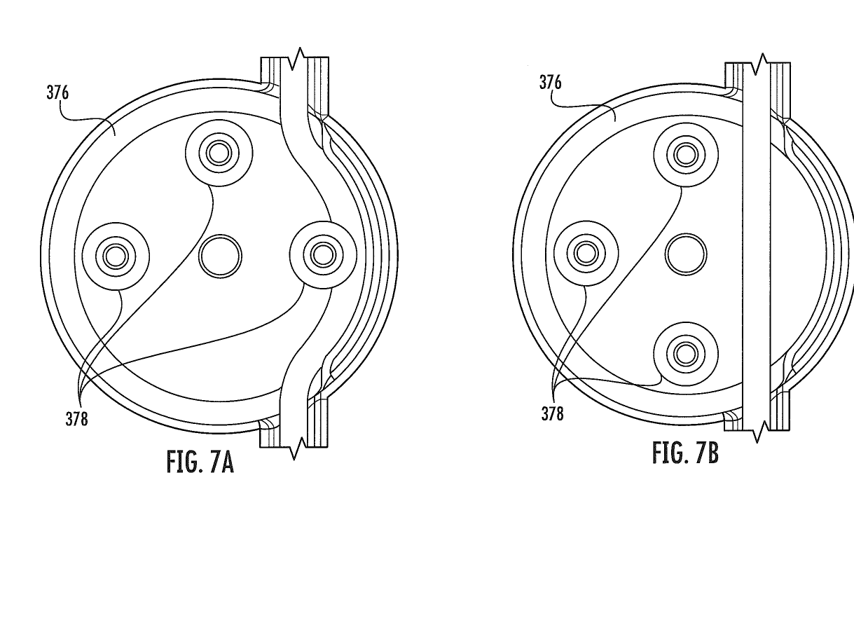

FIGS. 7a and 7b illustrate an alternative embodiment to the flow controller 20 that employs a three roller system. The flow controller 376 employs three rollers 378 for contacting the IV line to effect flow control. FIG. 7a illustrates a closed valve state, and FIG. 7b illustrates an open valve state for the flow controller.

Notably, the flow controller 20 of the illustrated embodiment employs a combination of the head pressure (primarily, except for the short draw and infusion by pinch point advancement) generated by the elevation of the fluid bag 32 on the pole 34 and the on-off regulation of the flow induced by the head pressure. The flow controller 20, however, could also include a combination of an actual powered pump and its programmable controller. This pump could be combined with the aforementioned calibrant solution source 16. One advantage, however, of the illustrated embodiment is that the gravity feed of the fluid bag 32 on the pole 34 is well-understood and mediated to control the amount of fluid administered to the patient. Use of active pumps should be controlled in some manner to avoid administration of excess fluid and its side-effects. Regardless, the role of the flow controller 20 can be met flexibly with various combinations of technology and the present invention shouldn't necessarily considered limited to any one particular configuration.

When the flow controller 20 opens its pinch valve, solution from the bag 32 is gravity fed down through the flush line 22, the sensor casing 24, the adapter 26, the blood draw line 28 and (if used) the catheter 30 and into the patient's vasculature. Or, the flow controller 20 could advance the pinch valve in the direction of the catheter 30 and drive the solution to flush the sensor 40 and out through the catheter. If the solution from the bag 32 includes heparin or other anti-thrombogenic agent and/or some anti-thrombogenic mechanical qualities, this flush step clears the catheter and cleans the sensor 40.

In a draw step, the pinch valve is reversed by the flow controller 20 forming a vacuum and drawing a blood sample up into the catheter from the patient's vasculature. The glucose sensor 40, during or after this step, can then be activated to sense the glucose concentration in the blood sample. After sufficient time has elapsed to take one or more analyte measurements, the flush cycle is then run, typically in 5 to 10 minute cycles, as described above. This process of flush-and-draw is repeated over the life of the sensing system 10, or at least the life of the glucose sensor 40. The description above is a more general overview of the flush/draw process. Variations in the specifics of the flush and draw cycles and how they're adapted to work with the present system to avoid thrombosis, minimize flush and draw volumes and work with existing catheter configurations will be described in more detail below.

In an embodiment of the present invention, the flow profile preferably lasts for 5 to 7.5 minutes and delivers less than 500 mL of solution from the bag 32 over a 72-hour period. Also, the flow controller 20 preferably has improvements to ensure accuracy and repeatability of its control of fluid flow through the flow control system 18. For example, the above-described rollers may be accompanied by an encoder coupled with a stepper motor that provides redundant control of the roller head orientation. Also, there may be an air detection sensor distal to the roller head assembly that uses optical or ultrasonic sensing (an ultrasonic pulse) to detect gas or liquid conditions in the tube segment.

As shown in FIG. 2, in one embodiment of the present invention, the sensor casing 24 includes a threaded flange 46, a cylindrical body 48 defining an axial lumen 56 and a female connector 50. The sensor casing 24 preferably has a length sufficient to protect the length (approximately 2 cm in a preferred embodiment) of the wire electrode sensor 40, such as about 4 cm. If the sensor casing 24 is too short, the adapter 26 might also supply some protection.

The threaded flange 46 is molded on the proximal end of the sensor casing 24 and extends around the cylindrical body 48 as a thin annulus with threads defined around its outer surface. The flange 46 is configured to insert into a luer connector at a distal end of the flush line 22. Defined within the flange 46 is an annular receptacle 58 (an expansion of the axial lumen 56) configured to receive a male portion of the luer connector. Attachment of the threaded portions of the connector and flange 46 should form a fluid tight communication between the lumen of the flush line 22 and the sensor casing 24.

The sensor casing 24 also may include an annular seal which is an elastomeric sealing member that is configured to extend between, and is compressed by attachment of, the male end of the luer connector and the threaded flange 46. Such compression seals off the junction between the two components and blocks wicking of blood and flush solution between the two components.

The cylindrical body 48 extends from the threaded flange 46 to the distal end of the sensor casing 24 and ends at the female connector 50. The cylindrical body has an elongate cylindrical shape and supports on its outside surface (and may be integrally constructed with) the housing 44 containing the board 42 through which the wire electrode sensor 40 connects to the communication line 36 of the monitoring line. The housing 44 has an elliptical or cylindrical shape to fit the electronic connection board 42 and includes a wire mount 54 extending off at about a 30 degree angle with respect to the axis of the sensor casing 24. The wire mount 54 helps to secure the monitoring line (communication wires 36) from detachment from the board 42 and its angle is tailored to having the communication line 36 extend off along and away from the patient and may allow the communication line to be taped to the patient's arm or bedside against being pulled free.

The axial lumen 56, as shown in the embodiment of FIGS. 2.4, has a cylindrical shape with a constant diameter extending down to the distal end of the cylindrical body 48. Optionally, the cylindrical body may also include a sleeve portion that extends around the axial lumen 56 and has smooth and thrombo-resistant properties that are improved with respect to the rest of the sensor casing 24. For example, the sleeve may be a portion of polyurethane or nylon tubing that is press fit into the sensor casing 24 after it is formed.

The cylindrical body 48 also defines a port 60 through which the wire electrode sensor 40 extends into the axial lumen 56 for exposure to the blood samples drawn therethrough by the flow control system 18. The port 60 is preferably sealed in some manner (such as by an elastomeric valve or being embedded in the material of the cylindrical body 48) against leakage of the calibration fluid and the blood samples and, in addition, is selected to smoothly integrate with the surrounding surface of the cylindrical body 48 that defines the axial lumen 56.

The axial lumen 56 preferably has a diameter that is selected to provide a smooth transition with the lumen of the flush line 22 and has sufficient space to fit the diameter of the wire electrode sensor 40. Embodiments of the present invention with variations of the diameter of the axial lumen 56 that achieve the objectives of providing for robust blood parameter sensing and minimized draw/flush volumes and thrombosis will be explored more below. However, for the illustrated embodiment, the diameter of the wire electrode sensor 40 is about 0.008 to 0.010 inch and the inside diameter of the axial lumen 56 is about 0.030 inch, which matches up for a smooth transition with a 0.030 inch lumen diameter of the flush line 22.

The female connector 50 at the distal end of the sensor casing 24 has a cylindrical shape with an outer cylindrical wall 64 spaced from an inner cylindrical wall 66 to form an annular female receptacle. The outer cylindrical wall 64 can include threads to enable attachment to a threaded proximal end 68 of the adapter 26. The inner cylindrical wall 66 extends within the proximal end 68 of the adapter 26. The positioning of these two walls brackets the threaded proximal end 68 of the adapter 26 for a firm connection between the two.

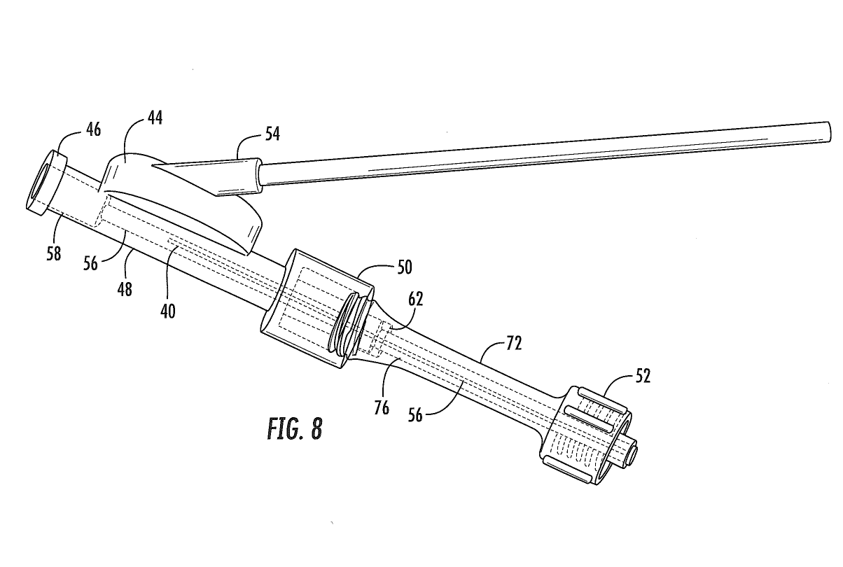

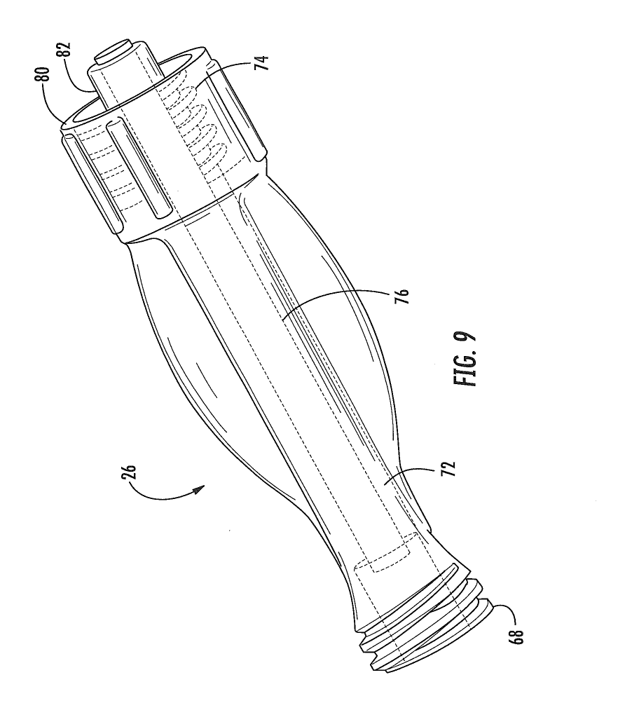

As shown in FIGS. 8 and 9, the adapter 26 includes the threaded proximal end 68, an annular seal 62, a cylindrical body 72 defining an axial lumen 76 and a threaded distal end 74. The threaded proximal end 68 is formed on the end of the adapter 26 and extends around the cylindrical body 72 to form a male fit with the female connector 50 at the distal end of the sensor casing 24. In particular, the threaded proximal end 68 has a threaded cylinder shape that extends between the outer and inner cylindrical walls 64, 66 of the distal end of the sensor casing 24.

The cylindrical body 72 extends from the threaded proximal end 68 to the distal end of the adapter 26, ending at the threaded distal end 74. The cylindrical body 72 has an elongate cylindrical shape and in another embodiment may include, as shown in FIG. 9, for example, fins 78 projecting outwardly from its surface to act as a stiffener against bending of the adapter 26. The fins 78 flare outwardly in a radial direction as they extend axially toward the threaded distal end 74 of the adapter 26.

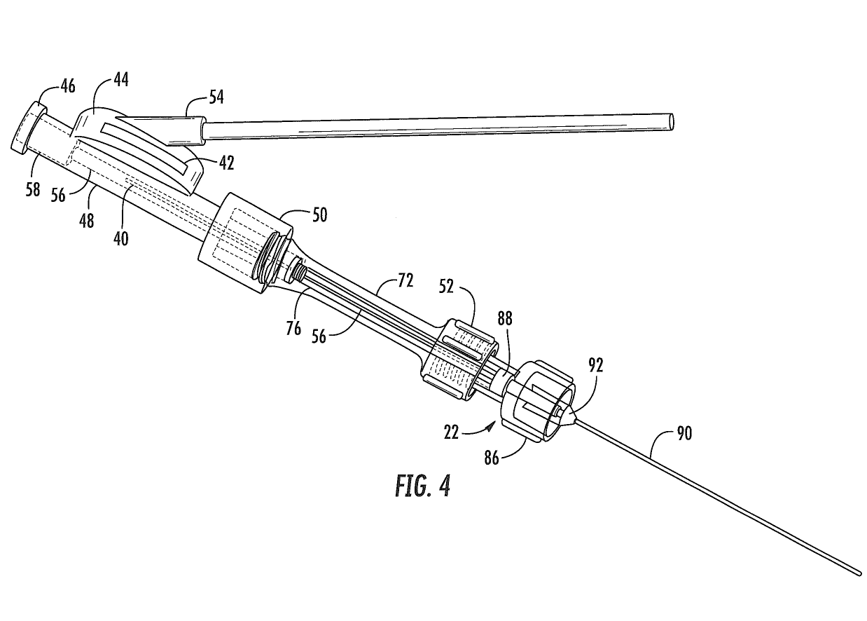

The threaded distal end 74 is fashioned similar to a luer connector with a pair of concentrically positioned, cylindrical outer 80 and inner 82 walls. The cylindrical outer wall 80 has threads extending around its inside surface that is configured to mate with a threaded proximal end 84 of the blood draw line 28. The cylindrical inner wall 82 projects more distal than the outer wall 80 and is configured to extend into the proximal end 84 of the blood draw line 28, as shown in FIGS. 3 and 4.

The axial lumen 76 defined by the cylindrical body 72 of the adapter 26 is configured to accept a free end of the wire electrode sensor 40. The length of the axial lumen 76 is just slightly longer, such as within 0.05 mm to 2 mm (preferably about 1 mm) the length of the wire electrode sensor 40. In this manner, the axial lumen 76 is configured to accept and allow extension nearly to its end the remaining length of the wire electrode sensor 40. The annular seal 62 is an annular elastomeric tube with a flange that is configured to fit within an expanded proximal end of the axial lumen 76 so as to seal against any leakage between the mating of the sensor casing 24 and the adapter 26.

Alternatively, the entire length of the axial lumen may be defined by a length of separately manufactured tubing press fit into the remainder of the adapter 26 which is formed as a molded part. This has the advantage of avoiding the difficulties of ensuring tight tolerances of the axial lumen 76 within the molded adapter 26. Ends of the tubing may extend out (e.g., 0.015 inch) of the surrounding opening within the cylindrical body 72 so as to enable a sealing fit at either of the proximal or distal ends 68, 74 of the adapter 26 when connected to the sensor casing 24 and blood draw line 28. Exemplary tubing may be 0.031 inch ID and 0.093 inch OD tubing with lumen clearance for 0.015 inch OD sensor wires, as shown in FIG. 3.

Similar to the axial lumen 56 of the cylindrical body 48 of the sensor casing 24, the axial lumen diameter can vary within ranges depending upon several factors associated with operation of the flow control system 18. However, for the illustrated embodiment, the diameter of the axial lumen 76 is preferably about 0.30 inch which provides 0.020 inch clearance around the end of the wire electrode sensor 40 extending therethrough.

Referring again to FIGS. 2, 3 and 4, the blood draw line 28 includes the threaded male proximal end 84, a locking cap 86, an axial lumen 94, a sealing member 88, a sampling line 90 and stress relief member 92. The proximal end 84 has a male shape configured to fit between the walls 80, 82 on the distal end 74 of the adapter 26. It also includes threads that fit the threads of the distal end 74 to secure it thereto in locking engagement. The locking cap 86 at the other, distal end has threads enabling it to fit the male end of a standard luer connector on standard catheters.

Defined through the proximal end and locking cap 86 is the axial lumen 94. Axial lumen 94 is enlarged on the proximal end and necked down through the middle and distal portions to a smaller diameter. The sealing member 88 extends within the axial lumen 94 and is an elastomeric member that has a tightly-toleranced inner diameter configured to fit an outer diameter of the sampling line 90, so as to secure the sampling line to the rest of the blood draw line 28. The sealing member also acts to seal the connection, through its elastic compressibility, between the adapter 26 and the blood draw line 28. The face of the threaded distal end 74 of adapter 26 abuts and compacts the flanged portion of the sealing member 88 when the male proximal end 84 of the blood draw line is twisted into the threads of the distal end 74. The flanged shape of the sealing member 88 secures against axial migration. Also, the sealing member 88 helps to secure the sampling line 90 to the rest of the blood draw line 28.

Also helping to secure the sampling line 90 is the stress relief member 92, which may be a dab of elastomeric adhesive in a frustoconical shape (as shown in FIG. 3) which helps to lock the sampling line to the sealing member 88 and/or the distal end of the locking cap 86 of the blood draw line 28. Or, the stress relief member 92 may be a length of tubing that has a decreasing diameter along its length to help relieve strain on the sampling line 90.

The sampling line 90 in one embodiment is a very small ID tube that has a relatively large OD and is constructed of a material that's mechanically thromboresistant (and may be combined with heparin or other anti-thrombosis agents) due to its internal shape, smoothness and void-free structure. Without being wed to theory, it is believed that the smaller ID is less prone to clotting or other thrombosis since the pressure profile across the cross-section of the blood is more evenly distributed because the red blood cells and other blood components are a larger percentage of the cross section of the lumen defined therethrough. More even pressure distribution helps to ensure that the blood components do not stop against the side of the lumen walls of the sampling line 90, cutting down on the tendency to clot. In addition, the smaller ID reduces the size of the flush and draw amounts to minimize side effects on the patient. Less blood in the draw means lower flushing volumes with the heparin in the calibration solution.

The relatively larger OD of the sampling line 90 is advantageous in that it provides a good buckling stiffness to enable insertion of the sampling line 90 directly into the patient (preferably in combination with a needle or other introducer) or into the lumen of an existing catheter 30 without bending or kinking. Still, if such a combination is desired, the OD can be constrained to allow the blood draw line 28 to be combined with existing catheters or introducers. In one embodiment, for example, the blood draw line has an outer diameter of 0.030 inch configured to fit within a range of standard-sized catheter 30 lumens, such as the three-lumen MULTI-MED central venous catheter or an ADVANCED VENOUS ACCESS (AVA) catheter (Edwards Lifesciences, Irvine, Calif.). Despite the aforementioned preferred configurations and sizes, a balance may be struck between a range factors, flow rates, adaptability to existing catheters, anti-thrombotic attributes and the ID/OD, length and other attributes of the sampling line 90 to create other embodiments of the present invention as will be described more below.

The advantage of inserting the sampling line 90 into an existing catheter 30 is that a dedicated line for sampling the analyte or blood parameter is no longer needed. In addition, the sampling line 90 can reduce the cross-sectional area through which blood is drawn to reduce clotting and sample volume. Further, the sampling line 90 can serve as a sleeve that covers the gaps, transitions and other voids that are present in conventional catheters.

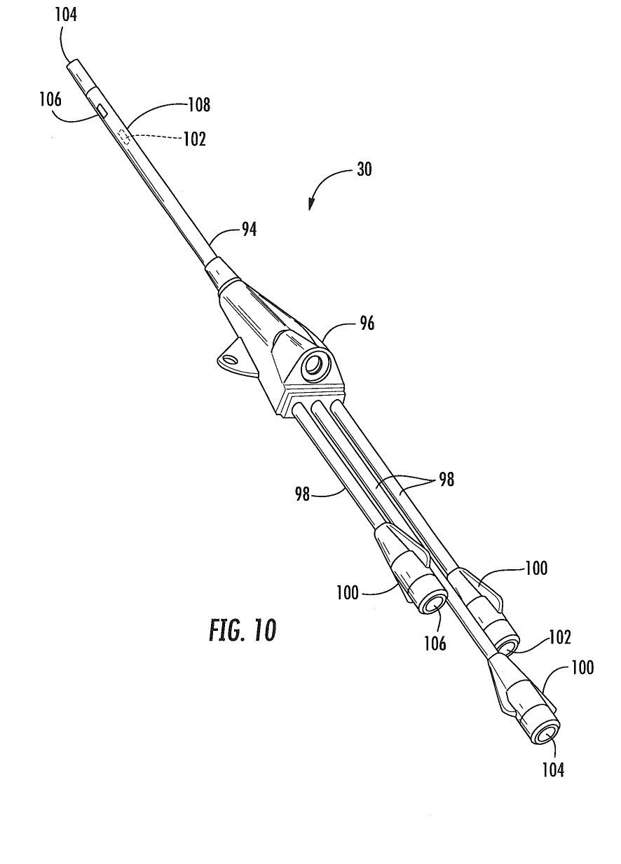

Conventional catheters 30, for example the catheter shown in FIG. 10, frequently include three parts, a multi-lumen 94, a backform 96 and lines 98. The multi-lumen 94 inserts into the patient and provides lumens that exit at different points of the multi-lumen tube depending upon the function employed with each lumen. For example, one lumen may be a supply lumen 102 for administering drugs that exits at the distal end of the lumen 94, another sensing lumen 104 for communicating with a pressure sensor for determining cardiac output that exits at a midpoint from the side of the lumen 94 and a third sampling lumen 106 for sampling blood that exits at a proximal point 108 from the side of the lumen 94.

Each of the lumens within the multi-lumen tube communicates with a dedicated channel defined in the backform. These channels diverge within the backform 96 (which typically has a triangular shape as it extends away from the patient) and each of the channels connects up with a dedicated one of the lines 98. Each time a transition between the components 94, 96, 98 occurs, there are discontinuities, gaps, rough surfaces, material variations and other voids that might promote the occurrence of clotting and other thrombosis and/or require less-desirable flow rates for the long-term, high-count sampling needed for the present invention.

In one embodiment of the present invention, the blood draw line 28 connects, via the locking cap 86, to a leer lock 100 mounted on the proximal end of one of the lines 98 that communicates through the backform 96 with the sampling lumen 106 of the catheter 30. The sampling line 90 extends through the line 98 and the backform 96 and partially through the sampling lumen 106, stopping about 1 inch short of the proximal exit point 108. Advantageously, the proximal exit point avoids draw of blood samples diluted or otherwise affected by the operations being performed in the other lumens 102, 104. Also, the sampling line 90 provides a void-free lumen that bypasses the voids formed by the junctions between the components 94, 96, 98, and the varied internal contours of those components, so as to reduce clotting and the volume of blood draws needed to supply the sensor 40. Stopping short of the proximal exit point 108 avoids extension of the sampling line 90 out of the exit port and making contact with the patient's vasculature.

As another alternative, the sampling line 90 may be of sufficient length to extend out of the exit point 108. This embodiment has the advantage of extending the void-free internal diameter of the sampling line past any irregularities at the end of the sampling lumen 106.

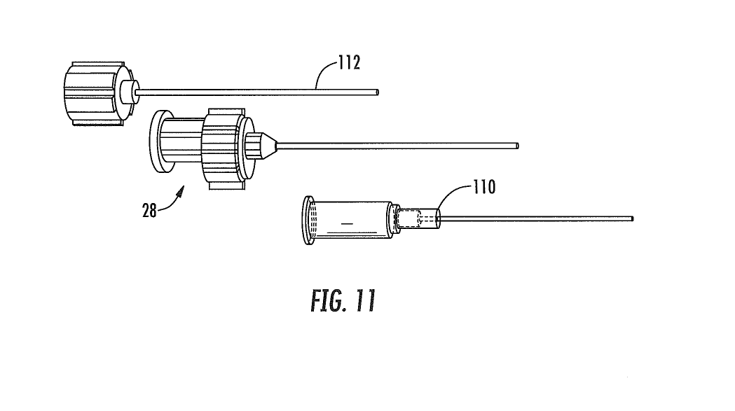

As another embodiment of the present invention, as shown in FIG. 11, the blood draw line 28 may be employed with its own dedicated introducer 110, such as the JELCO PROTECTIV SAFETY I.V. catheter which is a 20 gauge, 1.25″ long nylon catheter (Cardinal Health, Dublin, Ohio). In this embodiment, the blood draw line 28 may include a guide wire 112 that extends through the lumen of the sampling line 90 in order to give it additional stiffness. And, the sampling line 90 and guide wire 112 are inserted into the I.V. catheter, which is in turn inserted into the patient vasculature. The guide wire 112 may then be removed from the sampling line 90 and the proximal end 84 of the blood draw line 28 connected to the adapter 26, sensor casing 24 and flush line 22.

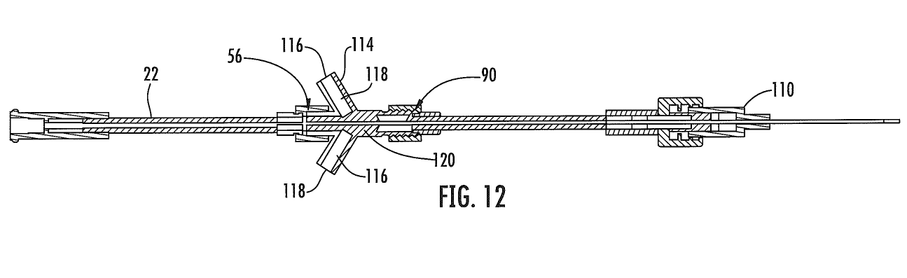

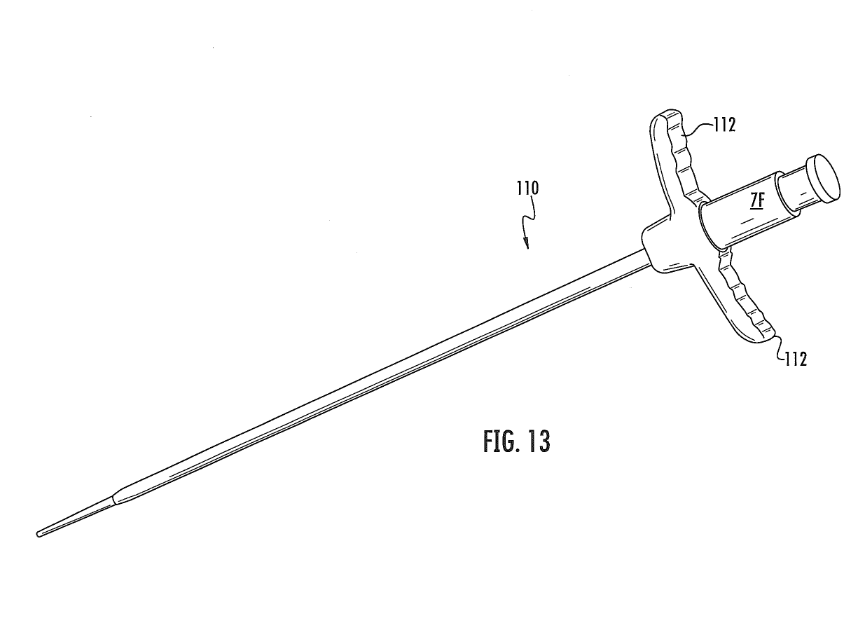

In yet another embodiment, as shown in FIG. 13, the dedicated introducer 110 may be a tear away introducer, such as the 3 French, 7 cm long GALT tear away introducer (Galt Medical, Inc., Garland, Tex.). The components are combined as described above, but after insertion two tabs 122 are pulled apart to split the catheter longitudinally and allow post-insertion removal of the dedicated introducer 110. Thus, only the sampling line 90 remains in the patient after the introducer is torn away. In this embodiment, the sampling line 90 is preferably of sufficient length to extend out of the end of the tear away introducer 110 so that it is less likely to be pulled out during tearing. As another alternative, the blood draw line 28 may also be connected to the adapter 26 and sensor casing 24 prior to insertion into the patient vasculature.

The length of the sampling line 90 can be selected based on a range of factors. In the embodiment described above, the sampling line 90 is configured to end about an inch short of the proximal exit point 108. This is because the variations in length of conventional catheters within a model can be relatively high (+/− inch) from the backform 96 through the extension lines 98. Longer length sampling lines 90 may be required for peripherally inserted central catheters (PICC), and could be 40 or even 60 cm long. Alternatively, the sampling line 90 could be much shorter and only extend past those regions of the catheter 30 with thrombosis generating qualities, such as past the junction between the backform 96 and the lumen 94 or whichever catheter regions are expected to be most prone to thrombus formation. For example, the CVC catheter may be 13.4 inches long but the sampling line 90 only 1.97 inches long. Shorter sampling lines 90, however, may use a two-stage blood draw process wherein the blood is first drawn into the catheter 30 and then later drawn into the blood draw line 28.

The length of blood draw line 28 (and adapter 26) could be selected on the proximal end to ensure a protective guard for the sensor 40. Also, the length of the sampling line 90 could be selected for ensuring sufficient durability of the combined blood draw line 28 and catheter 30, or could be selected to provide sufficient area for application of an anti-clotting coating. Lengths could also be varied to fit standard catheter 30 model lengths, allowing a healthcare worker to select and couple the catheter with the blood draw line 28 at the time of insertion. Lengths can range for CVC's from 16 inches, 20 inches and 30 inches, for example. Other lengths are also possible for different types of access devices, such as PICC's and IV catheters and introducers.

In one embodiment, the sampling line 90 has a constant 0.010 inch ID and a 0.025 inch OD so as to fit a range of standard-sized catheters 30. Also, the OD might be even smaller, such as 0.15 inch with a 0.010 inch ID, but the ID may be scaled down to keep bending stiffness high, such as down to 0.008 inch. The dimensions of the sampling line 90 and blood draw line 28 need not be consistent through its entire length. For example, the ID of the sampling line 90 could be larger closer to its proximal end to match up better with the axial lumen 76 ID of the adapter 26. The lumen of the sampling line 90 need not be cylindrical in cross-section and could be elliptical or have intervening walls to define multiple, split lumens through which blood could be drawn simultaneously.

FIG. 12 illustrates another embodiment of the present invention, wherein the flow control system 18 further includes a hub 114 that defines a pair of flared receptacles 116 that are configured to receive and end of a thermistor and the female connector 50 at the distal end of the sensor casing 24. These flared receptacles 116 communicate through side lumens 118 to an axial lumen 120 defined centrally in a body of the hub 114. The axial lumen 120 is enlarged to mediate the difference in ID between the flush line 22 and the axial lumen of the sampling line 90 (in this embodiment, no adapter 26 is employed). For example, the axial lumen 120 of the sampling line 90 may have an ID of 0.012 inch while the blood draw line has an ID of 0.040 inch. Within the axial lumen 120 of the hub 114 is a frustoconical shaped wall that transitions smoothly between these two ID's. When attached, the wire electrode sensor 40 and/or the thermistor extend through the side lumens 118 and into the axial lumen 120.

As another alternative embodiment, the sampling line 90 may extend through several components up to the flow controller 20, or may be integrated into the sensor casing 24 or other portion of the sensor assembly 14. Also, the flow control system 18 in another embodiment may be modified to flush around the sampling line 90 when it is mounted in catheter 30 to ensure blood is cleared.

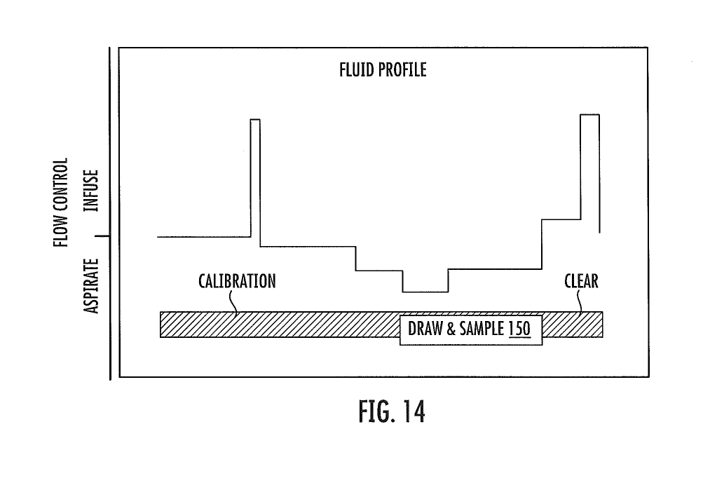

As shown in FIG. 14, the flow profile of one embodiment of the present invention includes a calibration and flush phase of about 276 seconds which includes 3.2 mL/hr for calibration, a flush of 650 mL/hr and trailing rates of 1.9 mL/hr and zero flow for a short time period. In the draw and sample phase, a 3.5 mL/hr draw is used with a zero flow rest period at the end. This is followed by the beginning of the flush phase with a 24 second “clear” flush using a 5 mL/hr start and then a ramped-up pre-calibration flush rate of 650 mL/hr.

In some embodiments, the system 10 may be employed over a 72 hour period and sample blood with 40 to 200 μL volumes in 5 to 10 minute cycles. With a 5 minute target blood glucose cycle and an approximate 90 second time window for draw volume, the maximum draw rate is about 200 mL/hour.

In some embodiments, there may be an advantage to making more frequent blood analyte measurements in a given time. For example, if there is instability with the patients analyte concentration or some other anomalies, additional readings can be taken in a time frame so that the sample measurements can be compared and either normalized or filtered to provide accurate readings. For example, wider draw volumes and/or more frequent blood draws could be employed (e.g., every 1 minute).

Although a range of materials may be used to construct the sampling line 90, polyurethane and nylon have shown experimental success. A factor, however, in selecting the material for the sampling line 90 is whether the material is transparent or translucent to the point of allowing visibility of blood from the draw cycle, which may impact patient morale. Therefore, opaque materials that mask the presence of blood may be desired, such as a green or opaque color.

FIGS. 2-4, 6 and 7 disclose a first embodiment of the analyte sensing assembly. As discussed above, during operation a blood sample is drawn into the assembly and over the sensor for analyte measurement of the sample. The blood sample is then flushed by a calibrant solution, such that the sensor is immersed in the calibrant solution for purposes of calibration. During analyte measurement, it is generally important to ensure that the sensor electrodes are presented with a blood sample that is not diluted by the calibrant. As such, an increase in the amount of blood drawn can ensure that a quality blood sample from the patient is presented at the sensor. As can be seen from the sensor assembly of FIGS. 2-4, 6 and 7, however, drawing additional blood from the patient may not be advantageous with this assembly, as the blood will travel along the sampling line 90 into a position where it is visible to the patient.

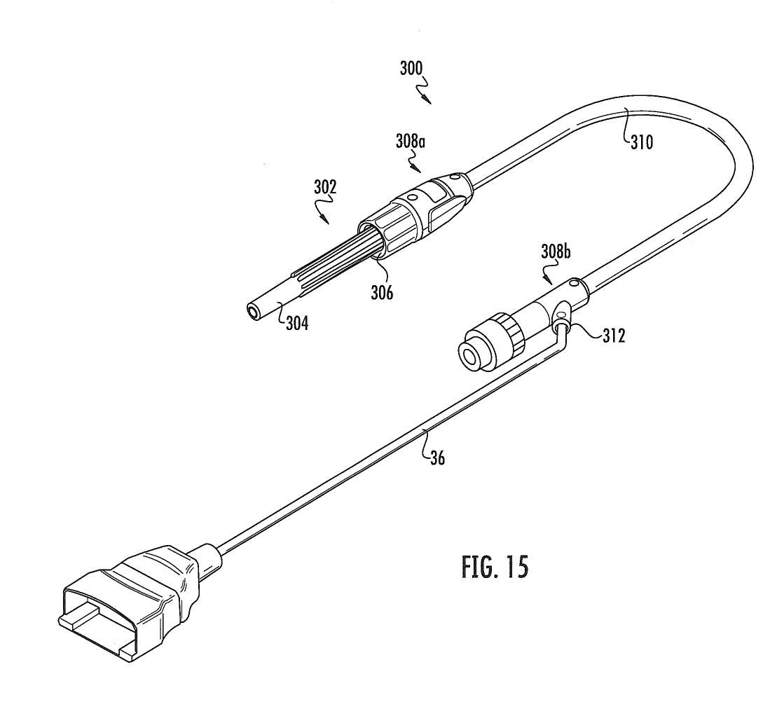

FIGS. 15-18 disclose an alternative sensor assembly 300 design. This sensor assembly design allows for increased blood sample draw. It also provides an electronic housing assembly that is somewhat modified from that of the sensor assembly of FIGS. 2-4, 6 and 7 by moving the location where the communication lines or wires are separated from the blood draw line. As illustrated in FIG. 15, the sensor assembly 300 of this embodiment comprises a sensor subassembly 302 for housing the sensor 40, not shown. In this depiction, a protective cap 304 with an associated luer 306 houses the sensor. The leads of the sensor are located in a first electronic housing subassembly 308a where they are connected to an electronics connection board discussed later below. In the electronic housing subassembly 308, the leads of the sensor are segregated from the blood draw line for connection to the monitor 12. Connected to the first electronic housing subassembly 308a is a multi-lumen blood draw line 310 comprising separate lumens for the monitoring line (communication lines or wires 36) and sampling tubing containing calibrant solution. The multi-lumen blood draw line 310 is connected at an opposed end to a second electronic housing subassembly 308b, where the communication lines are again separated at a wire mount 312 for extension to the monitor 12 of FIG. 1.

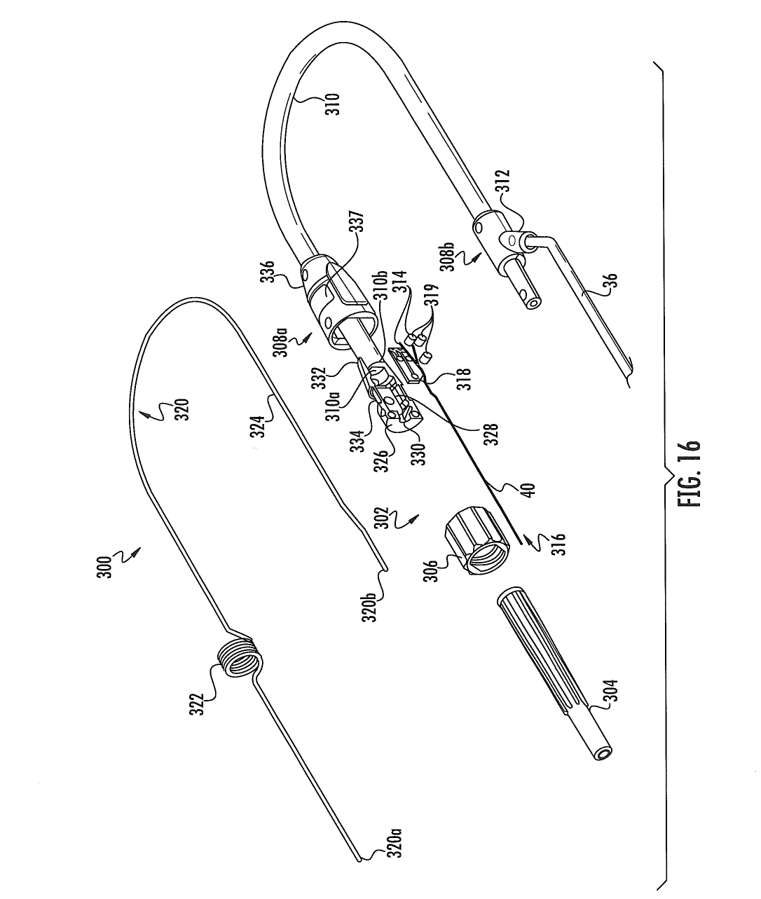

FIG. 16 is an exploded view of the sensor assembly 300 of FIG. 15. The assembly 300 includes a sensor 40 as described previously. The sensor 40 includes leads 314 on an opposite end from the electrodes 316 (e.g., working, counter and/or reference electrodes) of the sensor 40. The leads are connected to the communication lines or wires 36 via an electronic connection board 318. Respective, electrical conductive pucks 319 electrically connect the leads of the sensor to vias on the electronic connection board 318, which includes respective electrical traces for electrical connection to the communication lines or wires 36.

FIG. 16 also illustrates a perspective view of the end of the multi-lumen blood draw line 310. As illustrated, the multi-lumen blood draw line comprises a first lumen 310a and a second lumen 310b. The first lumen 310a is sized to receive the monitoring line (communication lines or wires 36) leading from the electronic connection board 318. The second lumen 310b is sized to receive the sampling line 320, as discussed later.

The sensor assembly 300 includes a modified sampling line 320 extending between opposed ends 320a, 320b. The portion of the sampling line proximate to end 320a that surrounds the sensor 40 is similar to the previously disclosed sampling line 90 discussed with reference to FIGS. 2-4. However, the sampling line 320 has an added coiled portion 322 and a distal elongated portion 324 extending to end 320b. The distal elongated portion 324 is designed to fit within the second lumen 310b of the multi-lumen blood draw line 310 and span the length of thereof. The modified sampling line 320 further includes an intermediary coiled portion 322 defining a selected length of tubing that can be coiled to reduce the amount of containment space needed to house the tubing. The coiled portion 322 of sampling tubing provides added volume for retention of the blood sample. This allows the system to draw an added volume of blood during sampling to ensure that the blood present at the electrodes of the sensor is not diluted by calibrant. The length of the coiled portion 322 of the sampling line 320 can be selected to be any desired length. Typically, the length from distal tip to the start of the coiled portion 322 is about 60 mm for a peripheral IV catheter. In one example, there may be 4-8 coils, or 4-7 coils, or 4-6 coils, with each coil being about 20-30 mm in length. Thus, in one example, the length of the tubing (from distal tip to a fluid connector) that when coiled comprises the coiled portion 322, is in the range of between about 150 mm to about 200 mm. In one example, the length of the tubing (from distal tip to a fluid connector) that when coiled comprises the coiled portion 322, is about 170 mm, and has 5 coils of about 25 mm each.

The first electronic housing subassembly 308a further comprises an insert 326 located within the subassembly. The insert 326 comprises a cavity 328 configured for retention of the electronic connection board 318. The cavity 328 includes an opening 330 at a first end configured to receive the body of the sampling line 320 and an opening 332 at a second end configured to mate with an end of the multi-lumen blood draw line 310, via, for example, a compression fit connection. The insert 326 further includes a cylindrical notched section 334 surrounding the outer periphery. The notched section 334 is configured to retain the coiled portion 322 of the sampling line 320. The notched section is sized so as to fit the wound coil of sampling line 320 in a compact manner in the subassembly 308a.

The first electronic housing subassembly 308a includes an outer housing 336 configured to maintain the insert 326, electronic connection board 318, and the coiled portion 322 of the sampling line 320. The outer housing 336 is configured for connection to the luer 306 and cap 304. In some embodiments, the outer housing 336 includes a reduced diameter opening for creating a compression fit between the opening 332 at a second end of the insert 326 and the end of the multi-lumen blood draw line 310. In some embodiments, the outer housing may include an opaque window 337 for viewing at least the coil portion of the sampling line.

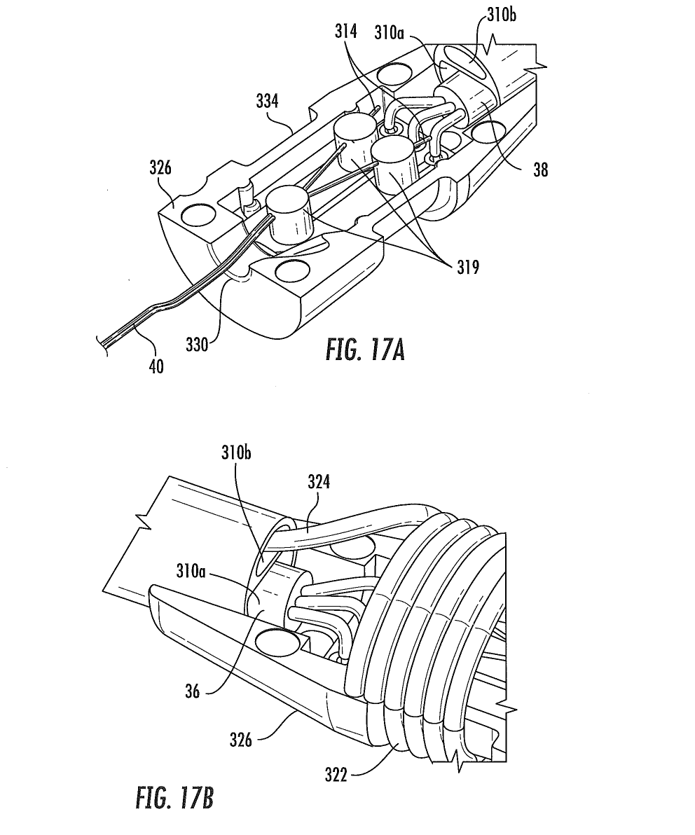

FIGS. 17a and 17b are exploded front views of a portion of the sensor assembly 300. In the depiction of FIG. 17a, the sampling line 320 has been removed. As viewed more closely in FIG. 17a, the leads 314 of the sensor are connected to electrically conductive traces 338 via the electrically conductive pucks 319. FIGS. 17a and 17b further illustrate the multi-lumen blood draw line 310 with the communication lines or wires 36 extending into the first lumen 310a, and the elongated portion 324 of the sampling line 320 extending into the second lumen 310b of the multi-lumen sampling line 310. The sampling line 320 is illustrated in a coiled configuration (portion 322) about the notched section 334 of the insert 326.

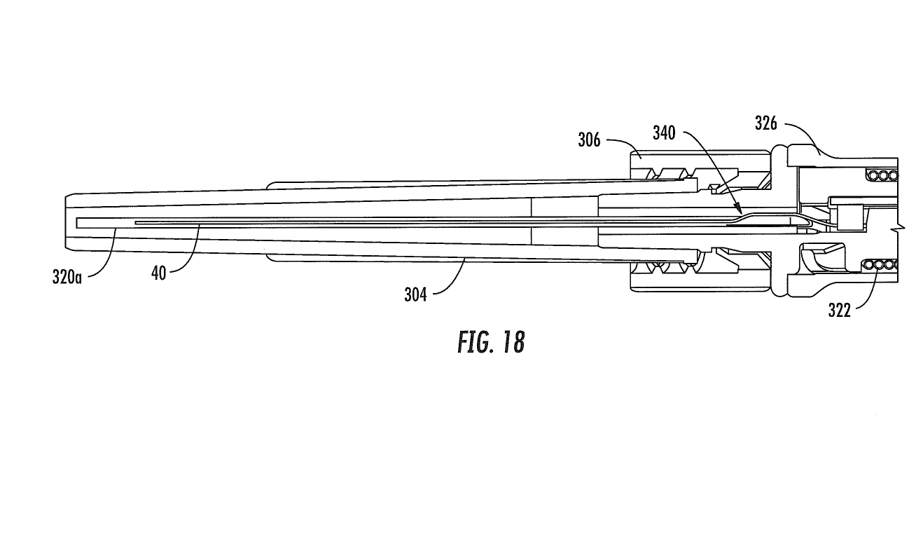

FIG. 18 is a cross-sectional view of a portion of the sensor assembly 300. FIG. 18 depicts the coiled portion 322 of the sampling line 320 coiled around the notched section 334 of the insert 326. Also illustrated is the exit of the sensor 40 from the sampling line 320 at location 340 prior to entrance in the opening 330 of the insert 326. The sensor 40 penetrates the side wall of the sampling line 320 thereby exiting the flow path for the blood sample and calibrant solution. The sampling line 320 sufficiently seals about the sensor to prevent leaking.

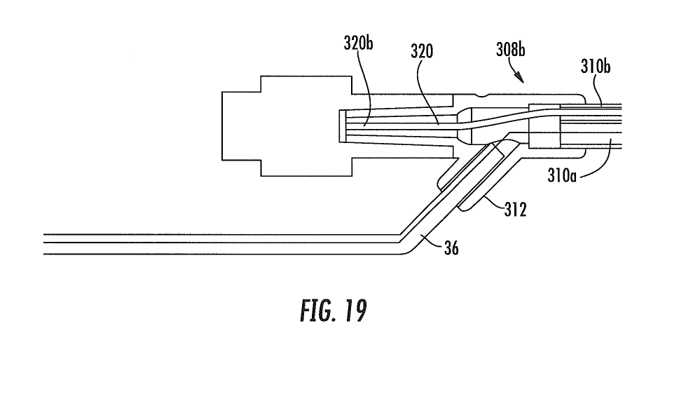

FIG. 19 is a cross-sectional view of the second electronic housing subassembly 308b of FIG. 12. The distal end 320b of the sampling line 320 extends from the second lumen 310b of the multi-lumen blood draw line 310 and terminates at a position within housing subassembly 308b. The communication lines or wires 36 exit the first lumen 310a and exit the second subassembly 308b via the wire mount 312 for extension to the monitor 12 of FIG. 1.

As discussed previously, the system includes a flow controller 20 for controlling the draw of blood samples and flush of calibrant. FIG. 20a depicts an embodiment of the system with a monitor 12 housing the flow controller 20. FIG. 20b depicts a portion of the flow controller 20 within the monitor 12. A cassette 350 as illustrated in FIG. 20c is employed to connect the flush line to the monitor 12. The cassette connects the calibrant solution from the calibrant bag 32 to the flush line 22. The body of the cassette is keyed for easy insertion into the flow control portion of the monitor. The cassette includes a safety mechanism in the form of actuator to prevent flow of calibrant from the bag or drawing of a sample from the patient under certain conditions. For example, when the actuator is engaged, it enables fluid to pass through and if relaxed prevents flow to “Fail Safe” when the door is opened or in the event of an instrument or power failure. The cassette may be shipped with the actuator in the relaxed position through use of a removable release tab. Without the release tab in place the actuator of the flow controller is in the relaxed position and shuts off flow. Upon insertion into the monitor, the monitor will control flow through the cassette.

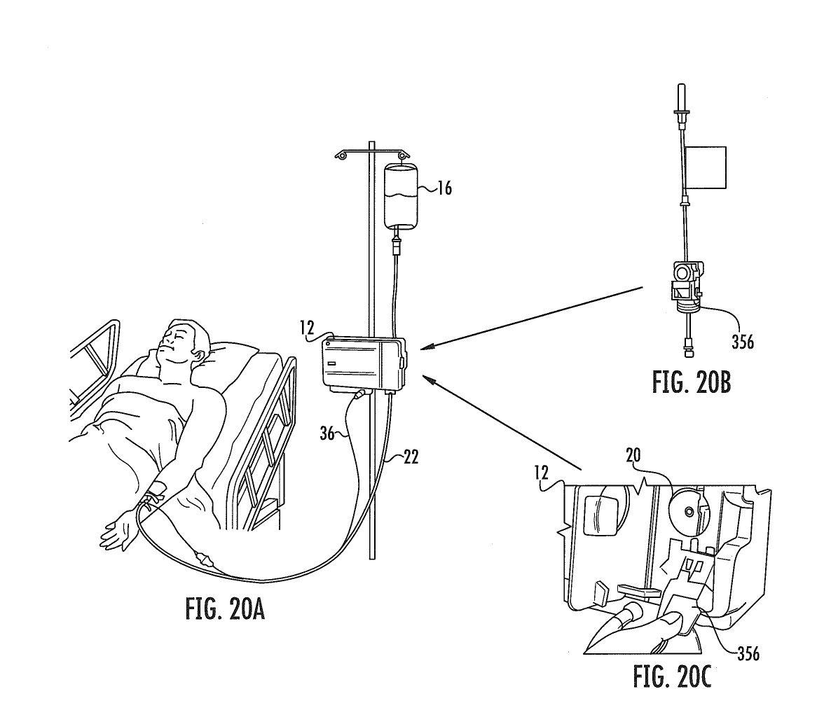

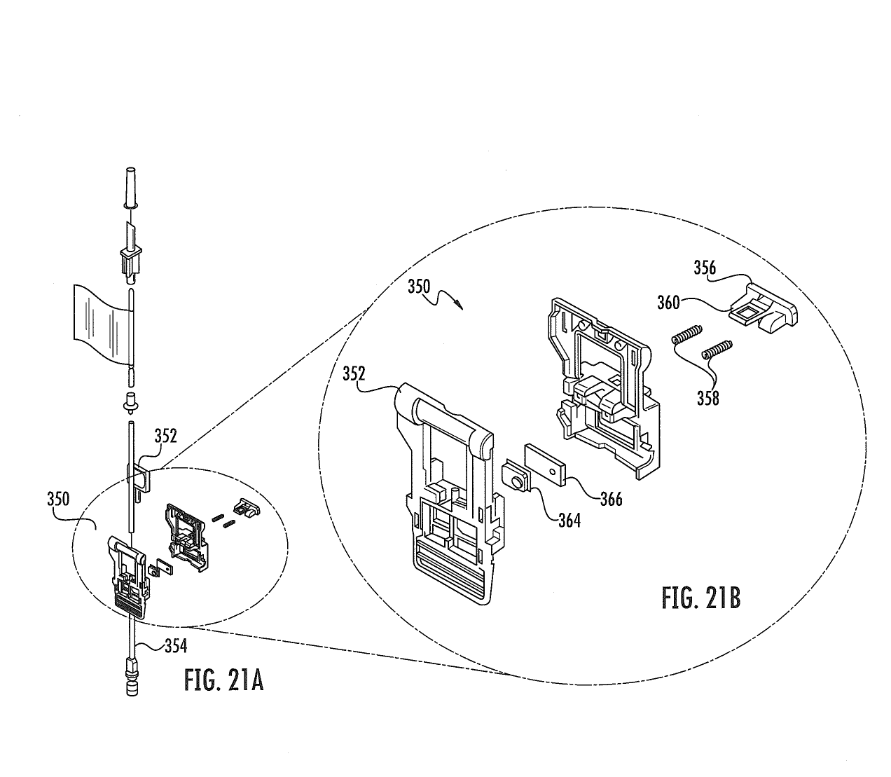

FIGS. 21a and 21b provide an exploded view of the cassette 350. The cassette includes IV lines and connectors for connecting respectively to the bag and the monitoring line. The cassette 350 includes a housing 352 that surrounds a portion of the IV line 354 extending therethrough. Associated with the housing is an actuator 356 and associated springs 358. The actuator 356 comprises an opening 360 that receives the portion of the IV line 354 extending through the cassette 350. The springs 358 bias the actuator 356 outwardly relative to the housing 352. In this biased position, an edge of the opening 360 of the actuator pinches or collapses the walls of the IV line, thereby restricting flow in the IV line. A release tab 362 may be provided for shipping purposes to maintain the actuator in an open (i.e., non-IV pinched position). Benefits of keeping the tubing in an un-pinched position is that it enables ethylene oxide (EO) gas into the tube for purposes of sterilization during the sterilization cycle. In addition, not pinching the tubing will prevent the tubing from getting occluded/pinched off before use. When the tab is removed, the springs bias the actuator to a pinched position. As an example, the door, when in the closed position, abuts the actuator of the cassette so as open the IV line. However, if the door were to be opened, the actuator would be biased outwardly closing the IV line.

With reference to FIG. 21b, in some embodiments, the cassette may include a pressure sensor 364 and associated electronics 366. The pressure sensor is placed in contact with the IV line extending through the cassette. The pressure sensor provides feedback regarding pressure within the IV line, which can be used to perform waveform analysis or detect potential line blockage, such as due to clotting, in the flow control path.

### EXPERIMENTAL

The present invention will now be described with specific reference to various examples. The following examples are not intended to be limiting of the invention and are rather provided as exemplary embodiments. An experiment was performed using a 4-roller flow controller 20. Porcine blood at about 37 degrees C. and 49% HCt mixed with heparin was drawn from a beaker as the “patient.” Two types of sampling line were used, a 0.008 inch ID PTFE tube and a 0.010 inch ID nylon tube.

The flow profiles used for the experiment included the following:

| | | | Exemplary | | |
|---|---|---|---|---|---|
| Profile | Profile | Rate | Cycle | | Volume |
| Phase | Stage | (mL/hr) | (Seconds) | | (mL) |
| Calibration | Bathe | 5 | 25 | | 0.035 |
| | Hold & sample | 0 | 257 | | 0.000 |
| | Sub total | | 282 | | 0.035 |
| Blood Draw | Draw volume | ~110 mL/hr avg | 100 | | −0.350 |
| | | Pre-Ramp-10 sec | | | |
| | | Ramp up-20 sec | | | |
| | | Steady rate-40 sec | | | |
| | | Ramp down-20 sec | | | |
| | | Slow Ramp down-10 sec | | | |
| | Hold & sample | 0 | 60 | Stabilization | 0.000 |
| | Sub total | | 160 | | −0.350 |
| Blood | Slow Clear | 0 | 0 | | 0.000 |
| Clear | Fast Flush | 1000 | 7.3 | | 2.028 |
| | Sub total | | 7.3 | | 2.028 |
| SUMMARY | | | | TOTAL VOLUME/CYCLE | 1.713 |
| | TOTAL | Seconds | 449 | TOTAL VOLUME/HR | 13.721 |
| | CYCLE | Minutes | 7.49 | TOTAL VOLUME/72 HR | 987.9 |
| | | | Exemplary | | |
| Profile | Profile | Rate | Cycle | | Volume |
| Phase | Stage | (mL/hr) | (Seconds) | | (mL) |
| Calibration | Bathe | 5 | 25 | | 0.035 |
| | Hold & sample | 0 | 230 | Stabilization | 0.000 |
| | Sub total | | 255 | | 0.035 |
| Blood Draw | Draw volume | ~110 mL/hr avg | 100 | | −0.350 |
| | | Pre-Ramp-10 sec | | | |
| | | Ramp up-20 sec | | | |
| | | Steady rate-40 sec | | | |
| | | Ramp down-20 sec | | | |
| | | Slow Ramp down-10 sec | | | |
| | Hold & sample | 0 | 60 | | 0.000 |
| | Sub total | | 160 | | −0.350 |
| Blood | Slow Clear | 0 | 0 | | 0.000 |
| Clear | Fast Flush | 1000 | 35 | | 9.722 |
| | Sub total | | 35 | | 9.722 |
| SUMMARY | | | | TOTAL VOLUME/CYCLE | 9.407 |
| | TOTAL | Seconds | 450 | TOTAL VOLUME/HR | 75.256 |
| | CYCLE | Minutes | 7.50 | TOTAL VOLUME/72 HR | 5418.4 |
| | | | Exemplary | | |
| Profile | Profile | Rate | Cycle | | Volume |
| Phase | Stage | (ml/hr) | (Seconds) | | (mL) |
| Calibration | Bathe | 5 | 25 | | 0.035 |
| | Hold & sample | 0 | 215 | | 0.000 |
| | Sub total | | 240 | | 0.035 |
| Blood Draw | Draw volume | ~110 mL/hr avg | 100 | | −0.189 |
| | | Pre-Ramp-10 sec | | | |
| | | Ramp up-20 sec | | | |
| | | Steady rate-40 sec | | | |
| | | Ramp down-20 sec | | | |
| | | Slow Ramp down-10 sec | | | |
| | Hold & sample | 0 | 60 | Stabilization | 0.000 |
| | Sub total | | 160 | | −0.189 |
| Blood | Slow Clear | 150 | 50 | | 2.083 |
| Clear | Fast Flush | 0 | 0 | | 0.000 |
| | Sub total | | 50 | | 2.083 |
| SUMMARY | | | | TOTAL VOLUME/CYCLE | 1.929 |
| | TOTAL | Seconds | 450 | TOTAL VOLUME/HR | 15.432 |
| | CYCLE | Minutes | 7.50 | TOTAL VOLUME/72 HR | 1111.1 |
| | | | Exemplary | | |
| Profile | Profile | Rate | Cycle | | Volume |
| Phase | Stage | (mL/hr) | (Seconds) | | (mL) |
| Calibration | Bathe | 5 | 25 | | 0.035 |
| | Hold & sample | 0 | 241 | | 0.000 |
| | Sub total | | 266 | | 0.035 |
| Blood Draw | Draw volume | ~110 ml/hr avg | 100 | | −0.189 |
| | | Pre-Ramp-10 sec | | | |
| | | Ramp up-20 sec | | | |
| | | Steady rate-40 sec | | | |
| | | Ramp down-20 sec | | | |
| | | Slow Ramp down-10 sec | | | |
| | Hold & sample | 0 | 60 | Stabilization | 0.000 |
| | Sub total | | 160 | | −0.189 |
| Blood | Slow Clear | 150 | 24 | | 1.000 |
| Clear | Fast Flush | 0 | 0 | | 0.000 |
| | Sub total | | 24 | | 1.000 |
| SUMMARY | | | | TOTAL VOLUME/CYCLE | 0.846 |
| | TOTAL | Seconds | 450 | TOTAL VOLUME/HR | 6.766 |
| | CYCLE | Minutes | 7.50 | TOTAL VOLUME/72 HR | 487.1 |

Pressure waveforms were recorded for blood draws and flushes over a 6 hour test of the above-described flow profiles and revealed consistent, repeatable results indicating no occlusions of the two tube types.

### Models

Many modifications and other embodiments of the invention set forth herein will come to mind to one skilled in the art to which this invention pertains having the benefit of the teachings presented in the foregoing description. As mentioned above, embodiments of the present invention balance different factors to achieve robust sensor measurements but to minimize the size of blood draw and flush volumes and to avoid thrombosis formation for long periods of time. For example, several key variables were analytically modeled by the inventors to determine their influence on performance of the flow control system 18 in a manner that stays within certain desired thresholds, such as a 10 psi maximum head pressure generated by the flow controller 20, while still allowing robust performance.

In a first model, the ID of the flush line 22 and the sampling line 90 were varied and the required pressures for each configuration were estimated. Assumptions of the structural components of the flow control system 18 included an 8 ft monitoring line with 0.012 ID; 1 cm long sensor with 0.35 mm diameter; 3 cm long casing 24 with 0.0314 inch ID; 34 cm long blood draw line 28 with 0.008 inch ID; catheter 30 lumen diameter of 0.030 inch with 1 inch length exceeding the distal end of the blood draw line 28; a 1.2 factor of safety for change in volume per cycle (maximum 200 mL/day and this is a minimization parameter, it should be as small as possible) on flush and a 3.0 factor of safety for the draw cycle volume (3 times the dead space of the CVC in front of the sampling line 90) plus the volume of the lumens from the tip of the sampling line 90 up to and around the sensor 40 to ensure a clean blood sample.

The assumed flow profile includes a 15 second time delay for mixing of the calibration solution after flush; a 100.539 second calibration flush at a 9.441 mL/hr flow rate; a 30 second hold at 0 mL/hr flow rate an 82.974 second blood draw at 3.468 mL/hr; another 30 second hold at 0 mL/hr and a blood flush of 41.487 seconds at 6.936 mL/hr.

Parameters calculated from these assumptions included 79.929 μL sample volume, 263.654 μL per cycle flush volume, 75.932 mL per day flush volume and a length of blood in the flush line of 2.033 ft. The ratio of flush to sample was 3.299. The pressure of the calibration flush was 7.482 psi, the blood draw 3.741 psi and for blood flush was 7.482 psi. All of these were in acceptable ranges, representing a feasible configuration. This model was verified against experimental testing results and observed pressures were all less than modeled maximum pressures, namely about 5.51 psi or 35% less for calibration flush, 3.67 psi or 1.8% less for blood sample draw and 6.65 psi or 12.4% for blood clear flush.

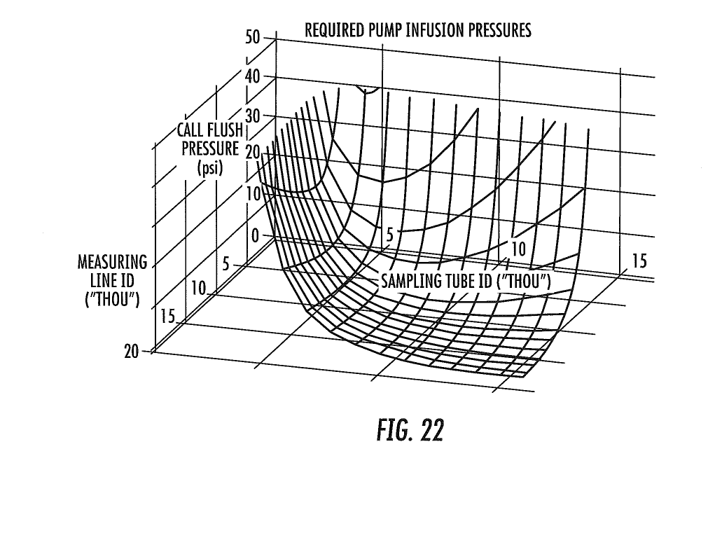

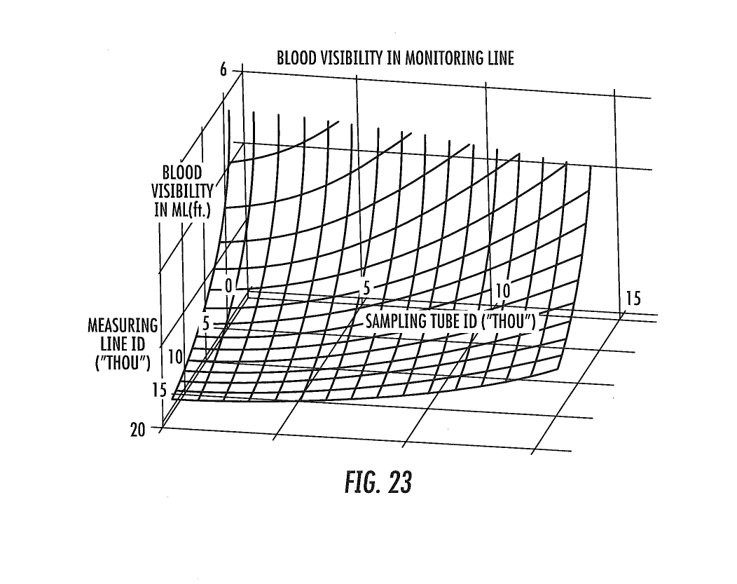

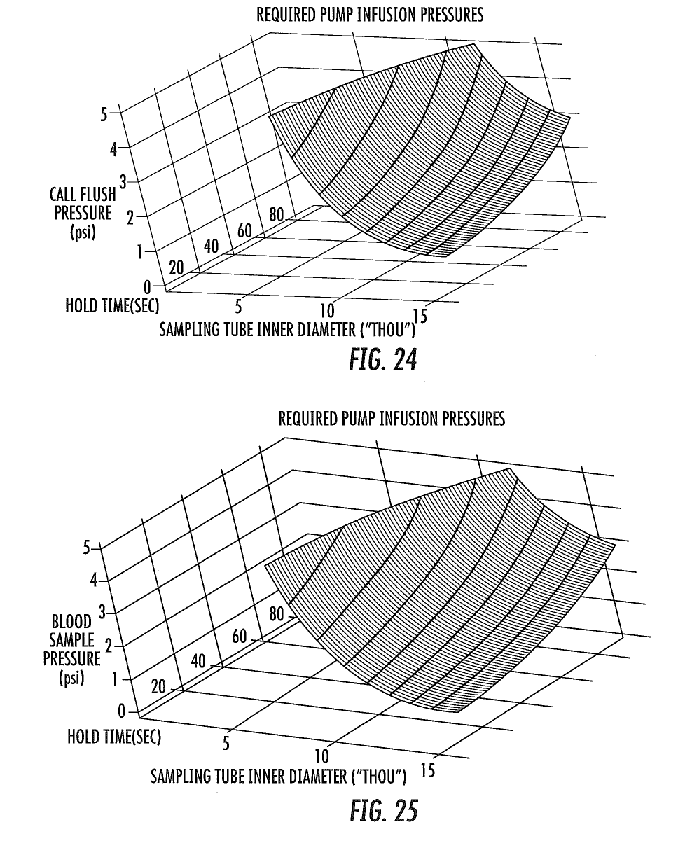

This model was then further explored by varying the ID's of the monitoring line 22 and sampling line 90 to determine the resulting flush pressure and length of visible blood in the monitoring line. The flush pressure is desirably below 10 psi due to size, cost and other constraints on the available power of the flow controller 20. Also, it is desirable to minimize the amount of blood visible in the monitoring line 22 to alleviate patient anxiety. Results of these models are shown in FIGS. 22 and 23.

A second analysis was run with modified assumptions of 0.011 inch ID monitoring line; 0.0066 inch diameter wire sensor 40; sample draw volume 70.182 μL, flush volume 53.861 mL/day for a 2.665 flush to sample volume ratio and 3.091 ft of blood in the flush line 22. The flow profile was modified by a calibration flush at 69.564 seconds at 9.678 mL/hr; sample blood draw for 103.624 seconds for 2.438 mL/hr and blood flush for 51.812 seconds at 4.876 ml/hr. The calibration flush pressure was 9.678 psi; sample blood draw pressure 4.839 psi and blood flush pressure was 9.678 psi. All values notably below the preferred 10 psi threshold for pump head pressure for the flow control system 18.

A third analysis was run to explore a lower pressure limit, below 6 psi for the flow controller 20. Flush line ID was 0.014 inch; sensor casing 24 ID was 0.014 inch; wire sensor 40 diameter was 0.0084 inch and sampling line 90 ID was 0.009 inch. Volume of blood sample was 80.428 μL; flush volume per day was 86.186 mL for a 3.721 flush/sample ratio and length of blood in the flush line 22 was 2.121 ft. The flow profile was modified to 86.025 seconds for calibration flush at 12.523 mL/hr; sample draw was 92.65 seconds at 3.125 mL/hr and blood flush was 46.325 seconds at 6.25 mL/hr. The resulting pressures were 5.77 psi for calibration flush; 2.885 psi for sample draw and 5.77 psi for blood flush, all below the desired 6 psi threshold.

A fourth analysis was run with the following changed assumptions: flush line ID 0.013 inch; sensor casing ID 0.0013 inch; wire sensor diameter 0.0078 inch and sampling line ID 0.010 inch. The blood sample volume was 89.722 μL; flush volume per day 74.549 mL for a 2.885 flush/sample ratio and 2.703 ft of blood in the flush line. The flow profile was 82.148 seconds, 11.344 mL/hr calibration flush; 95.234 seconds, 3.392 mL/hr blood sample draw and 47.617 seconds, 6.783 mL/hr blood clear flush. Results were 5.433 calibration and blood clear flush pressure and 2.716 psi blood draw pressure.

A full range of pump flush (infusion) and blood sample draw pressures was explored by varying the diameter of the sampling line 90 and the hold time for the respective infusion and draw hold times. The results are illustrated in Fs. 25 and 25.

In another embodiment of the present invention, it has been observed by the inventors in time periods shortly (or immediately) after a sensor's calibration or initialization and shortly (or immediately) following a period of unpowered disconnect that the sensor's sensitivity may be changing rapidly. This rapid change reduces the effectiveness of the sensor's sensitivity determined during calibration. Generally, the rate of sensitivity change is proportional to an error generated by the change during the period between calibration and testing.

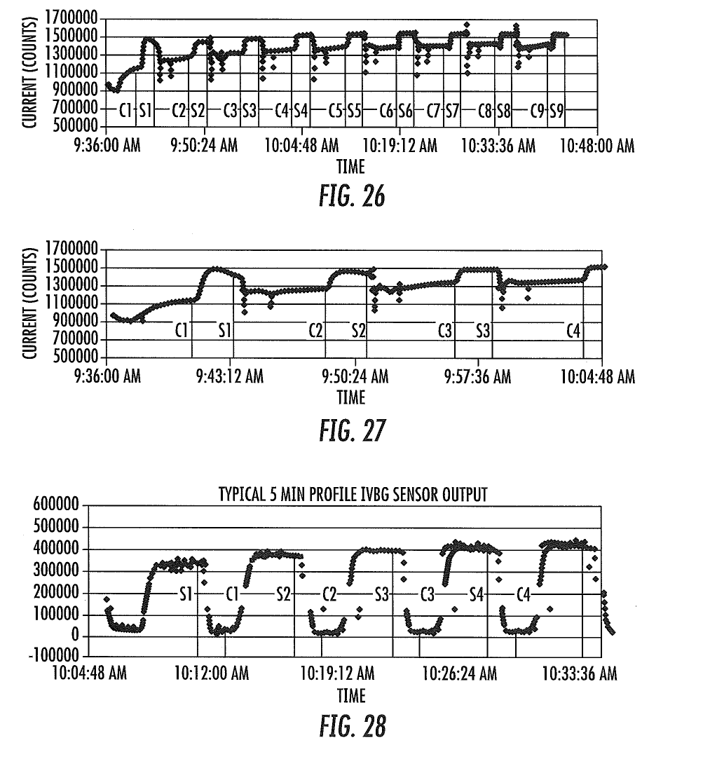

As shown in FIG. 26, calibration and sample pairs were taken with high sensitivity sensors using a 7.5 minute profile with a time gap between the calibration (C#) and sample (S#) phases of approximately 2.5 minutes. This data represents the time allowed for a sensor to “run-in”, which means the sensor has achieved a nominal level of stability.

As shown in FIG. 27, the sensitivity change is at a substantial enough rate that the sensitivity calculated during the calibration phase is not representative of the sensitivity during the sample calibration phase. In one embodiment, the present invention includes the use of a statistical method to estimate the sensitivity change between the calibration and sample. For example, a simple linear regression could be applied to the first two (or more) sensitivity calculations (vertical lines associated with C1 and C2) to estimate or interpolate the sensitivity change. Also, a logarithmic interpolation could be used. Further, leading or trailing data points could be used to model the sensitivity trends.

FIG. 27 shows that, absent such an interpolation, the sensitivity used at C1 has changed by the time the sample S1 is taken. Using the sensitivity from C1, therefore, produces an error in the estimated glucose value returned by the sensor algorithm. Conversely, such error is reduced through use of a statistical estimation of the rate of change of sensitivity as a function of time, and then use of the modified sensitivity to estimate the glucose concentration of the sample. The error reduction has been on the order of 1% to 10% using such techniques.

In another embodiment of the present invention, the drift in the sensor sensitivity can be reduced by shortening the time during which such drift can occur. For example, the time between C1 and S1 can be minimized to decrease the sensitivity change and allow for an improved accuracy of the calculated glucose concentration. The flow profile may be modified to minimize the time by reducing the entire profile length, such as from 7.5 minutes to 5 minutes. Further, the method may modify the order in which sample and calibration measurements are made. As shown in FIG. 28, for example, the sample value is calculated prior to the calibration value, which shortens the delay between sample and calibration to about 1.5 minutes.

Notably, the data indicated in FIG. 28 was taken with a 0 mg/dL calibration solution. Thus, the sensitivity drift is not readily apparent from the calibration phase, but is apparent from the sampling phase.

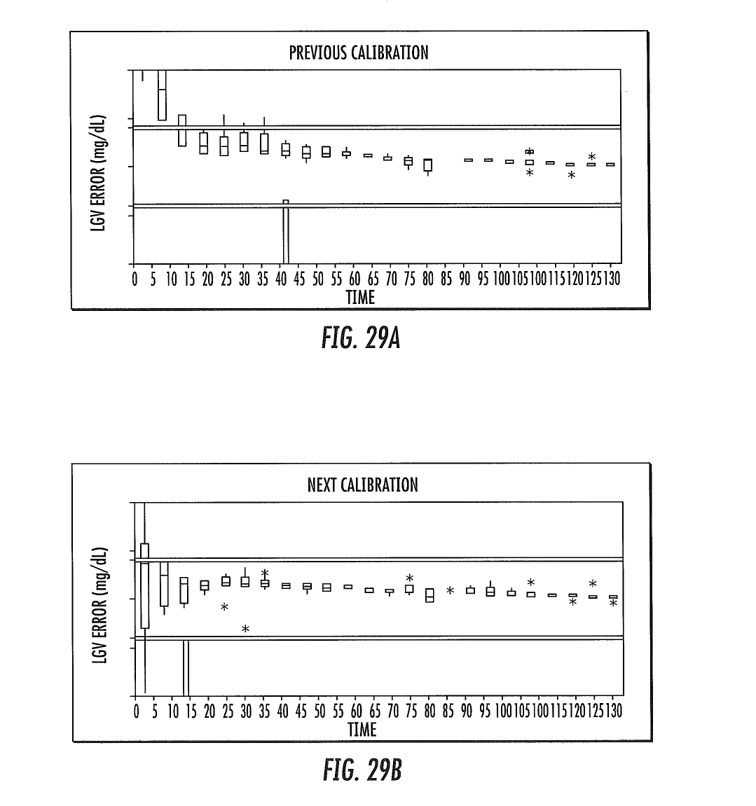

As shown in FIGS. 29a and 29b, use of a “next calibration” embodiment for a study population of n=8 with a 5 minute flow profile and high-sensitivity sensor decreased the error over a “previous calibration.” In FIG. 29a, the plot is “previous calibration” and the bottom plat (FIG. 29b) is “next calibration.” Notably, as indicated by the vertical black bar, the threshold for run-in time was reduced from about 40 minutes to 15 minutes using embodiments of the present invention.

In another embodiment, the rate of sensitivity drift could be predicted or modeled and the rate multiplied by the delay between sample and calibration.

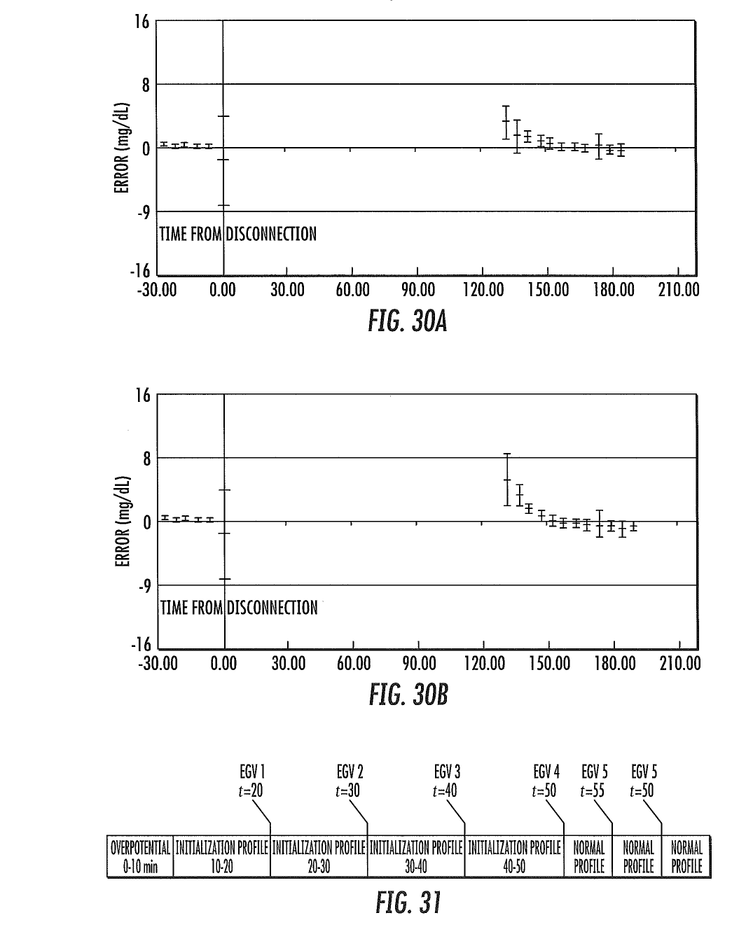

In another embodiment, the present invention may be used to improve accuracy of glucose concentration measurements after a “re-connect,” the period of time after a sensor has lost all power, such as would be seen after a patient transport. For example, data from a “re-connect” comparison are shown in FIGS. 30a and 30b. The top plot is using the “next calibration” which produces tighter measurements than the bottom plot using “previous calibration.”

Advantages of these embodiments include improvements in estimated glucose concentration accuracy, particularly during states in which the sensitivity of the sensor is changing rapidly. The improved accuracy is especially beneficial during the sensor initialization or “run-in” period and can significantly reduce the time required to reach accuracy thresholds. Notably, sensors for other types of blood (and biological) parameters, such as pH, pCO2, pO2, K+, Na+, Ca++, lactate and haematocrit, with drift or run-in periods may also benefit from embodiments of the present invention.

The inventors have also observed that continuous analyte monitoring systems employing “one size fits all” flow profiles may be unable to detect and adapt to flow problems. For example, obstructions, kinking of the blood access device or limited blood flow may create conditions where the flow profile is no longer adequate and sample dilution occurs. Detection of these flow problems is difficult because the signal from the sensor plateaus while the sensor is bathed in the sample. In another embodiment of the present invention, these problems are addressed by a method that continuously flows the blood sample past the sensor instead of bathing the sensor in a static blood sample. When the blood sample is always moving, assuming sufficient blood is drawn, plateaus formed in the sampling phase will be achieved only during specific conditions. Either the sensor is located within a homogeneous blood sample, a homogenously diluted sample or a calibrant fluid. A homogenously diluted sample, for example, may be found if the patient's blood flow is obstructed and the local venous environment is contaminated with calibration or flush fluid. This is a narrower set of circumstances than the above-listed problems causing non-homogenous sample dilution. Constantly moving the blood, therefore, can allow the plateau information occurring outside of fixed sample windows to be used to determine analyte concentration. In addition, the presence of the plateau ensures, with greater confidence, that the sample is either fully homogenous blood or calibrant fluid. Conversely, the lack of a plateau, or the time required for the plateau to develop from the initialization of fluid flow, can indicate problems with flow dilution. For example, over a 20 second interval of 0.5 Hz sampled data, the coefficient of variation could be limited to less than 5% or even less than 1%, or some increment in between. Also, or alternatively, it can be inferred that if the time to achieve a plateau is longer than the times previously achieved, either in an acute instance, or a slow degradation, different error modes related to flow can be inferred.

In another embodiment, a diagnostic mode may be employed to correct for errors indicated by the lack of a plateau. For example, mathematical waveforms may be employed to perform non-linear regression and recover lost/corrupt waveform data. Also, the model may be able to predict the plateau value.

Advantageously, continuously flowing the sample blood or calibrant enables the use of plateau information to diagnose problems with the sample or system. Also, the plateau information can be used to adjust the flow profile for greater calibration and sampling efficiency and accuracy.

In yet another embodiment, the present invention includes a method of in-vivo flow profile determination using a blood parameter sensor. Generally, the blood parameter sensor determines the appropriate pump flow characteristics to improve blood access device compatibility and reduce system setup time or cycle time. This is particularly advantageous for access devices with unknown parameters or automated detection of characteristics of the blood access device. This allows “plug and play” operability, sparing healthcare personnel from determining and communicating the particular characteristics of the blood access device supporting the analyte sensor.

As shown in FIG. 31, for example, an initialization phase employs an initialization flow profile. The initialization profile may be longer, shorter or the same time period as the nominal (default) flow profile and is used to determine blood volume, and other flow profile characteristics, customized to the access device. The initialization period may have more than one initialization profile, such as the four profiles shown in FIG. 31.

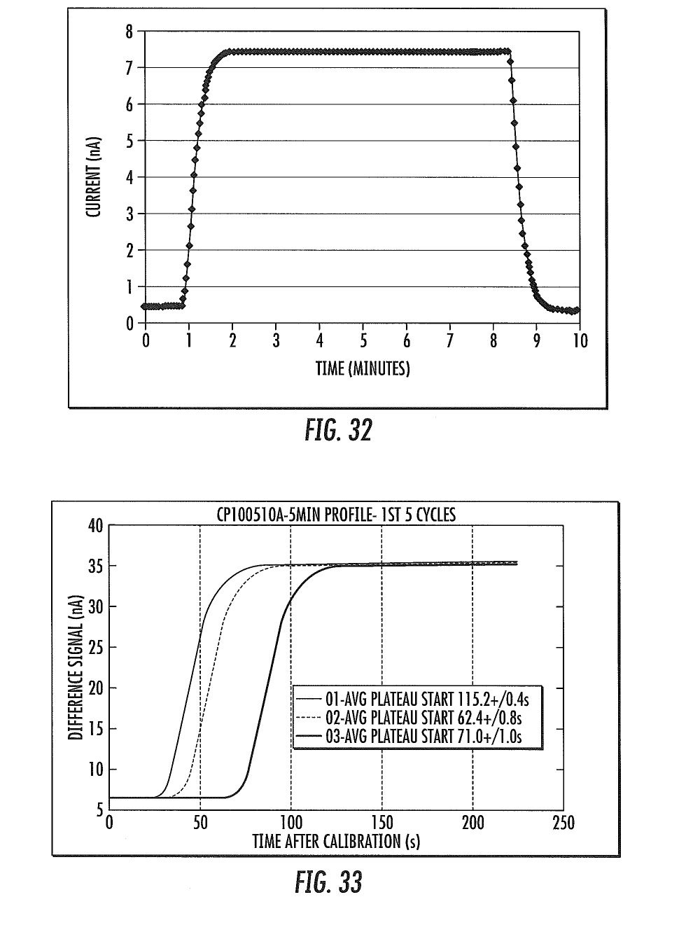

Regardless of variation in the aforementioned variables and the configuration of the access device, the initialization profile is preferably configured to have at least one maximal blood draw. For example, as shown in FIG. 32, a 10 minute initialization profile is configured to draw a 200 μL sample. In this example, the sensor output can be seen to change current at approximately 1 min and has stabilized by 2 minutes into the draw. As described in a prior embodiment, the time to reach complete immersion in sample fluid can be determined algorithmically by using plateau detection to identify a substantially stable period.

It should be noted that while the signal of the embodiment illustrated in FIG. 32 represents an actual glucose sensor trace, the signal can be from other sensors capable of blood detect, such as a thermal sensor (thermistor) which measures the change in temperature between the two fluids. Also, multiple signals from multiple sensors may be polled to produce a robust combined result.

In the initialization profile, the draw rates are preferably known and recorded during the entire cycle. The flow rate multiplied by the time to reach the plateau, or the average of the time measured over multiple profiles or devices, yields the blood draw volume for the particular system configuration and clinical setting.

In another embodiment, the initialization profile may itself be dynamic and determine the appropriate blood draw volume with improved accuracy by using a series of profiles where blood draw volume and flow rate are varied. For example, initialization profiles 1, 2, 3, and 4 shown in FIG. 31, have flow volumes and rates of 400 μL at 40 μL/min; 200 μL at 20 μL/min; 100 μL at 10 μL/min; and 50 μL at 5 μL/min. Use of several different initialization flow profiles increases accuracy of the blood draw volume by modeling a range of circumstances. The calculated blood draw volume can then be used to choose between a plurality of pre-determined flow profiles, such as through use of a lookup table indexing a database.

Alternatively, or additionally, the results of the initialization profiles may be used to customize the flow profile. For example, the custom profile may have a predetermined set of valve transitions to ensure a desired uniform sensor sample return rate. However, the required blood draw volume may still be achieved by changing the blood draw rate of the nominal profile to ensure sufficient blood draw volume, without changing the returned waveform or the plateau characteristics.

In another example, the custom profile may have a predetermined blood draw rate and the required volume may be achieved by changing the duration of draw of the nominal profile to achieve the desired blood volume. This method could be used to produce a uniform sensor sample return rate that may differ from the nominal return rate, but advantageously minimize cycle time needed for a particular access device.

As shown in FIG. 33, three different individual sensor configurations, including three different access devices, were tested in a sample initialization phase where 200 μL was drawn at a constant rate over 10 minutes. The three configurations include the same sensor located within (1) a prototype hub with a long blood draw line (sipper tube) as would be used in a central venous catheter, (2) a prototype hub with a short blood draw line (sipper tube) as would be used in a peripheral catheter, and (3) a dedicated peripheral catheter.

Notably, the differences in configuration affect the time at which initial blood detection and total immersion of the blood parameter sensor occur.

Advantages of the above-described embodiments of the invention include automated flow profile determination to improve compatibility with a range of sensor and access device configurations. Also, customized flow profiles may increase the sensor operation time. Also, the return rate of glucose concentration measurements by the sensor may be optimized or customized to improve the availability of information for clinical dosing decisions.

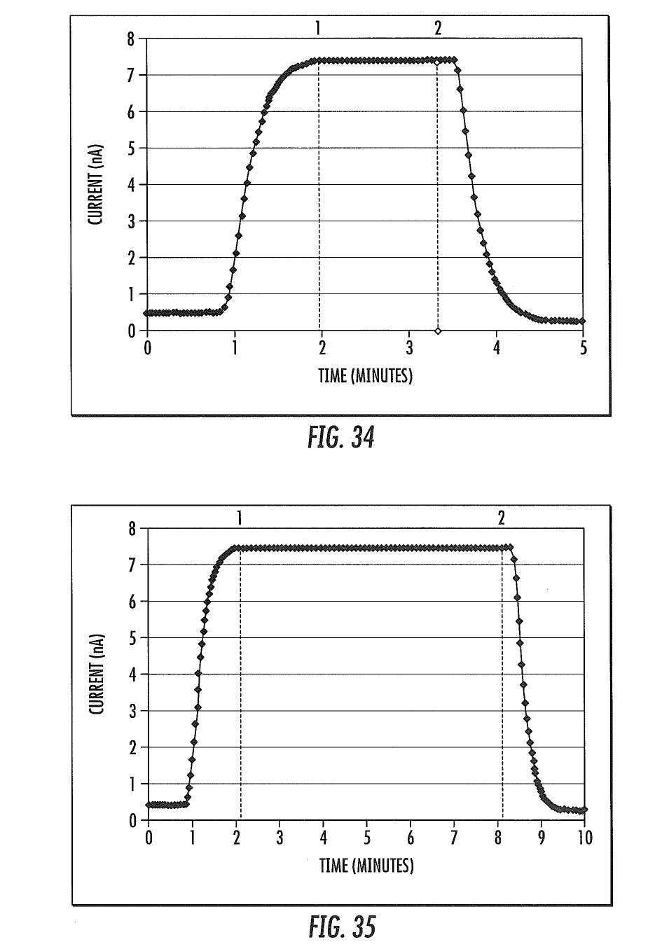

A flow profile of another embodiment of the present invention includes continually drawing blood into the sensing location so as to achieve higher resolution glucose measurements. For example, as shown in FIG. 34, the cycle time can be kept at 7.5 minutes, or shortened, while the blood is continuously drawn, allowing for a period of time (˜1.5 minutes) of continuous analyte determination, as defined as the period of time between 1 and 2. The flow rate at which blood is drawn may be held constant or increased to allow rapid blood exposure of the sensor. Using the above described embodiments, an algorithmic plateau detector and/or dedicated blood parameter sensor may be used to determine whether the blood sample is undiluted and a glucose value can be calculated. This glucose value can be immediately returned to the clinician without having to wait for an entire blood cycle to finish. And, as long as the blood is continually drawn into the sampling chamber, the glucose value can be continually updated. This provides true continuous monitoring of the patient's blood glucose value throughout regions 1 and 2, as shown in FIG. 34.

In one embodiment, the blood draw can be continued until a clinically relevant volume of blood has been withdrawn. The time-span for the blood draw may be increased beyond the nominal profile's 7.5 minutes to increase the continuous sampling portion of the waveform, as shown in FIG. 35 between regions 1 and 2.

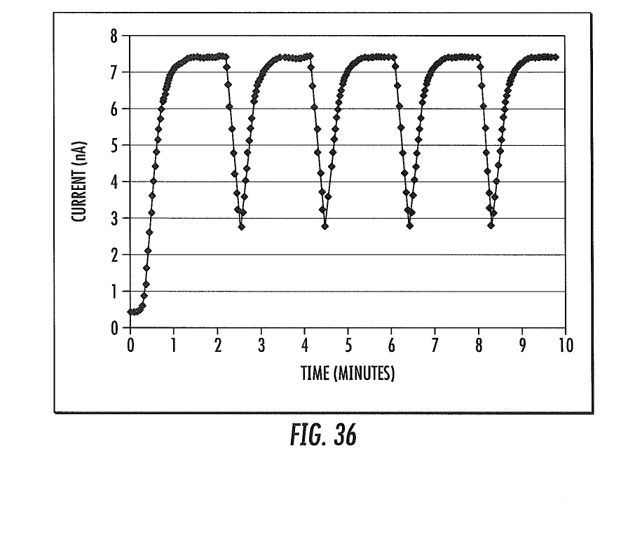

In some clinical scenarios it may be advantageous to avoid drawing an additional volume of blood from the patient into the sensor. In this case, another method for increasing the resolution of the measurement is to eliminate the calibration cycle for a pre-determined period of time, as shown in FIG. 36. In this embodiment, a glucose concentration value is calculated from an individual draw, and rapidly flushed from the system, at which point a new sample is immediately sampled.

Notably, in the embodiments of FIGS. 35 and 36, the time between calibrations is extended compared to a 7.5 minute default or nominal profile. This need not impact accuracy of the sensor as calibration may be bolstered by use of the previously described embodiments. The embodiments of FIGS. 35 and 36 are also valid for use when there is no glucose in the calibration solution, and the flush fluid is used solely to evacuate the blood from the sensor.

Advantageously, the extended blood draw and continuous sampling increases the resolution (compared to a single measurement every 7.5 minutes) of glucose measurements for the clinician. Increased resolution is advantageous in post-clinical interventions, when either glucose or insulin has been administered. During this period of time, the patient's actual blood glucose value may be changing rapidly, and higher resolution information may facilitate more precise clinical dosing, intervention or system algorithmic decisions. For example, higher resolution measurements could facilitate more accurate glucose value trend calculations.

Increased resolution would result in more accurate glucose value trend calculations. For example, the system could run profile to obtain several measurements in quick succession (e.g., 90 second intervals) to determine an obstruction or required draw volume for a particular device. It also could be used to bolster confidence in a particular glucose value if measured glucose value was outside the “physiologic limit” For example, the clinician just injected a large amount of glucose systemically to recover from a hypoglycemic event, and the resulting change is not physiologically possible. Also, it could be used to recover a value if the algorithm determined that the previous plateau was unacceptable due to noise or some other exclusionary criteria.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below (and above) with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

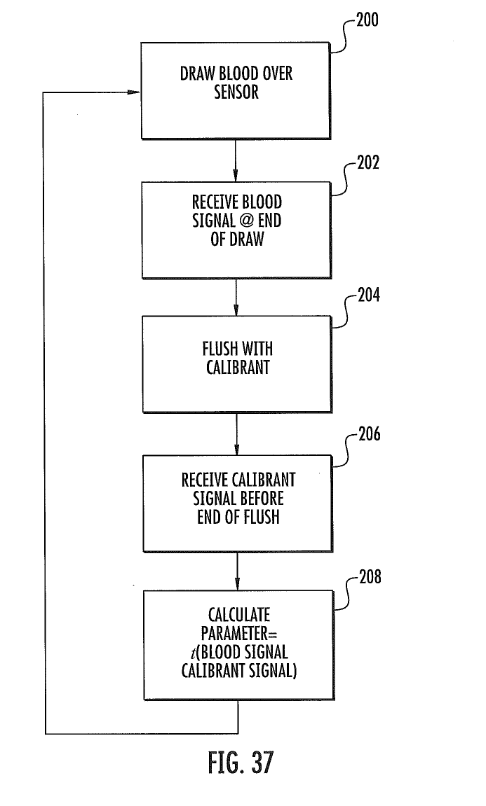

Other embodiments of the present invention may include systems, methods, processes or computer programs for calibrating a blood sensing system and/or operating a blood parameter sensor system. For example, as shown in FIG. 37, one embodiment of the present invention includes drawing blood 200 over a blood parameter sensor, receiving a blood signal 202 near the end of the draw, flushing the sensor with calibrant 204, receiving a calibrant signal 206 before the end of the flush and calculating a blood parameter 208 as a function of both the blood signal and the calibrant signal.

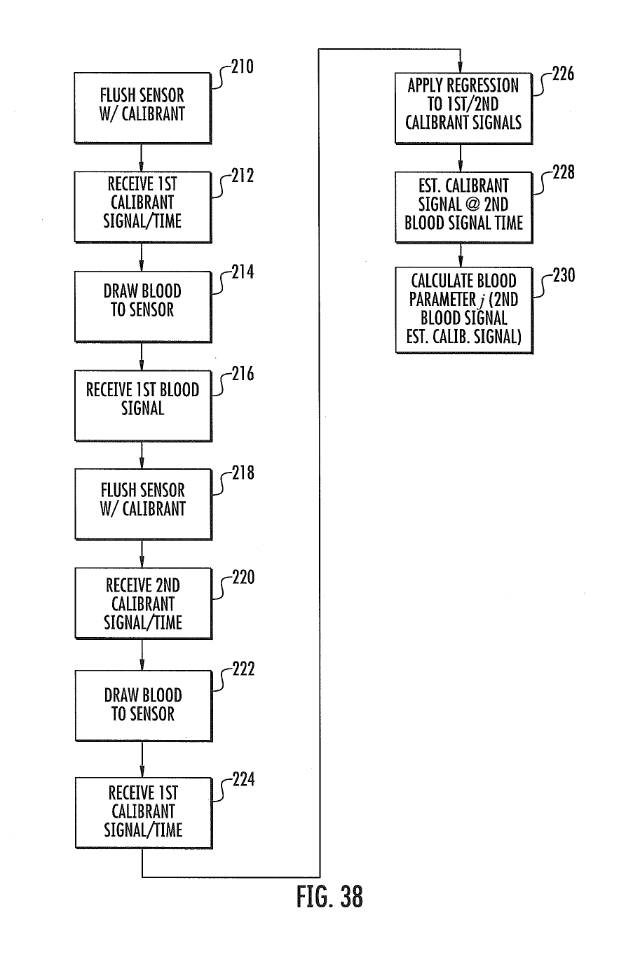

FIG. 38 shows another embodiment that includes flushing the blood parameter sensor with calibrant 210, receiving a first calibrant signal and time 212, drawing blood to the blood parameter sensor 214, receiving a first blood signal 216, flushing the blood parameter sensor with calibrant 218, receiving a second calibrant signal and time 220, drawing blood to the blood parameter sensor 222, receiving a second blood signal and time 224, applying a (linear or logarithmic) regression to the first and second calibrant signals 226, estimating a calibrant signal at the second blood signal time 228 and calculating a blood parameter as a function of the second blood signal and estimated calibrant signal 230. These embodiments may reduce the error due to sensor drift by modeling, and adjusting for, drift in the sensor calibration.

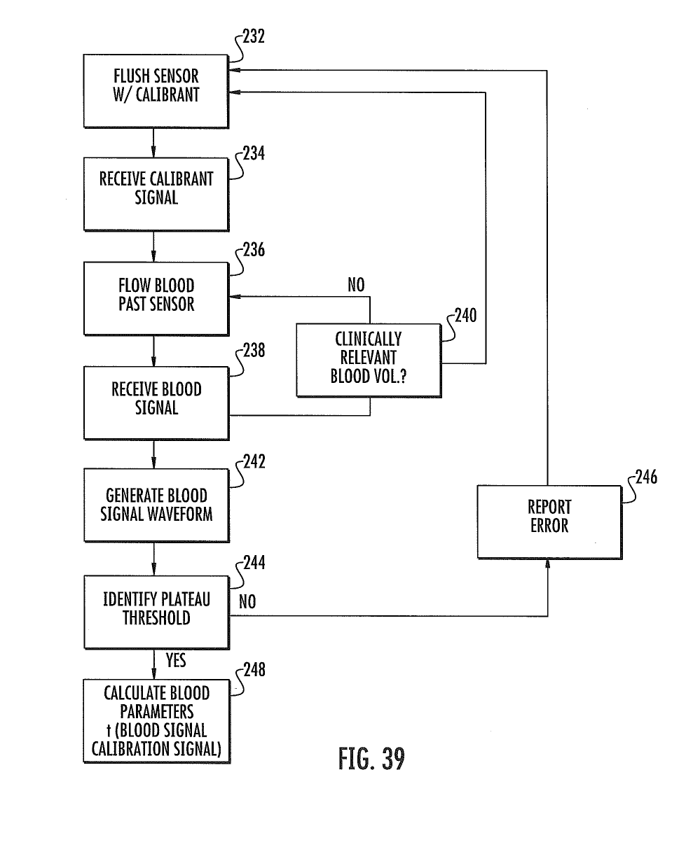

FIG. 39 shows another embodiment that includes flushing a blood parameter sensor with calibrant 232, receiving a calibrant signal 234, flowing blood past the blood parameter sensor 236, receiving a blood signal 238, repeating 236-238 continuously until a clinically relevant blood volume is reached 240, then generating a blood signal waveform 242, identifying a plateau threshold (if possible) 244 and calculating blood parameters as a function of the blood signal over the threshold and the calibration signal 248. If a plateau threshold cannot be identified and the clinically relevant blood draw volume limit has been reached 244, the system would report an error 246 and/or restart the cycle by flushing the blood parameter sensor with calibrant 232. Advantageously, using a threshold to determine when the blood signal is accurate reduces reporting errors.

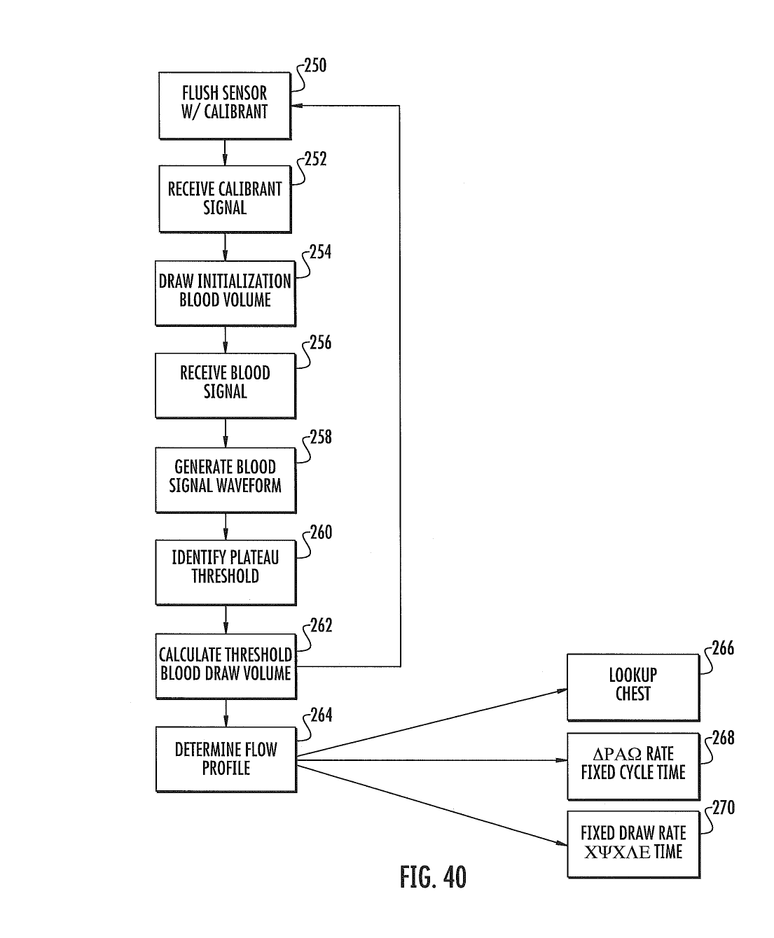

FIG. 40 shows another embodiment that includes flushing a blood parameter sensor with calibrant 250, receiving a calibrant signal 252, drawing an initialization blood volume 254, receiving a blood signal 256, generating a blood signal waveform 258, identifying a plateau threshold 260 on the blood signal waveform, calculating a threshold blood draw volume 262, repeating, if desired, steps 250-262 to obtain several threshold blood draw volumes, and determining flow profiles 264 for nominal operation using the threshold blood draw volume(s). Determining flow profiles 264 may include, for example, looking up the flow profile in a chart or database 266, changing the draw rate to achieve the threshold volume in a fixed cycle time 268, and/or, with a fixed draw rate, changing the cycle time 270 to achieve the threshold volume. Advantageously, these embodiments adapt to unknown blood access configurations that may be combined with the sensor and flow control system.

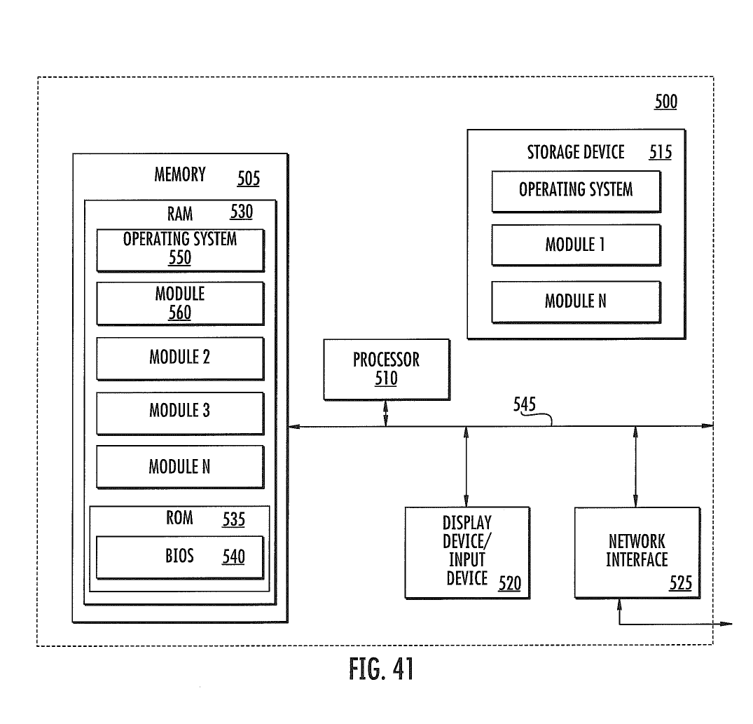

Referring now to FIG. 41, a schematic diagram of a central controller 500, or similar network entity, configured to implement a blood parameter sensing system, according to one embodiment of the invention, is provided. As may be understood from FIG. 41, in this embodiment, the central controller 500 may include a processor 510 that communicates with other elements within the central controller 500 via a system interface or bus 545. Also included in the central controller 500 may be a display device/input device 520 for receiving and displaying data. This display device/input device 520 may be, for example, a keyboard or pointing device that is used in combination with a monitor. The central controller 500 may further include memory 505, which may include both read only memory (ROM) 535 and random access memory (RAM) 530. The server's ROM 535 may be used to store a basic input/output system 540 (BIOS), containing the basic routines that help to transfer information across the one or more networks.

In addition, the central controller 500 (such as a combination of the monitor 12 and flow control system 18) may include at least one storage device 515, such as a hard disk drive, a floppy disk drive, a CD Rom drive, or optical disk drive, for storing information on various computer-readable media, such as a hard disk, a removable magnetic disk, or a CD-ROM disk. As will be appreciated by one of ordinary skill in the art, each of these storage devices 515 may be connected to the system bus 545 by an appropriate interface. The storage devices 515 and their associated computer-readable media may provide nonvolatile storage for a central server. It is important to note that the computer-readable media described above could be replaced by any other type of computer-readable media known in the art. Such media include, for example, magnetic cassettes, flash memory cards, digital video disks, and Bernoulli cartridges.

A number of program modules may be stored by the various storage devices. Such program modules may include an operating system 550 and a plurality of one or more (N) modules 560. The modules 560 may control certain aspects of the operation of the central controller 500, with the assistance of the processor 510 and the operating system 550. For example, the modules may perform the functions described above and illustrated by the figures, such as FIGS. 37-40, and other materials disclosed herein.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As is evident from the range of modeled and experimentally verified embodiments described above, the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A blood access system for use with a system for sampling blood from a patient's vasculature and directing the blood to a blood parameter sensor, the blood access system comprising:
a blood draw member with a proximal end and a distal end;
the blood draw member defining at least one lumen extending from the proximal end to the distal end;
wherein the distal end of the blood draw member resides within the patient's vasculature for an extended period of time; and
wherein the proximal end is connectable in fluid communication with the blood parameter sensor;
the blood draw member including an inside surface that defines the lumen and is smooth, continuous and void-free so as to avoid clotting;
the lumen of the blood draw member including an inside diameter of less than 0.025 inches so as to avoid clotting during flush and draw pressures of up to 10 psi.

2. A blood access system of claim 1, wherein said blood draw member comprising a coiled portion proximate said proximal end, such that said coil portion increases the overall length of the blood draw member.

3. A blood access system of claim 1, wherein the inside diameter of the blood draw member is within a range of 0.008 inches to 0.015 inches.

4. A blood access system of claim 3, wherein the blood draw member includes an outside diameter within a range of 0.015 inches to 0.025 inches.

5. A blood access system of claim 4, wherein a difference between the inside and outside diameters is at least 0.005 inches.

6. A blood access system of claim 4, wherein a difference between the inside and outside diameters is at least 0.015 inches.

7. A blood access system of claim 4, wherein a ratio of the outside to inside diameters is at least 2.5 times.

8. A blood access system of claim 1, wherein the inside surface defines axial structure configured to improve kink resistance.

9. A blood access system of claim 1, wherein the blood draw member has a buckling strength sufficient to withstand direct insertion into the patient's vasculature and subsequent movement of the patient's appendage.

10. A blood access system of claim 1, wherein blood draw member comprises at least one of a polyurethane or polyimide.

11. A blood access system of claim 1, wherein the blood draw member comprises a nylon.

12. A blood access system of claim 1, wherein the inside diameter increases as the lumen extends from the distal to the proximal end of the blood draw member.

13. A blood access system of claim 1, wherein the inside diameter is constant from the distal to the proximal end of the blood draw member.

14. A blood access system of claim 1, wherein the blood access member defines a plurality of lumens.

15. A blood access system of claim 1, containing a lumen that is smooth, continuous and void-free so as to avoid clotting during blood draw or flush.

16. A blood access system of claim 1, wherein the blood draw member is configured to be clot-free up to a maximum draw rate of 200 mL/hr.

17. A blood access system of claim 1, wherein the blood draw member is configured for connection at its proximal end into fluid communication with a monitoring line and to receive a fluid solution from the monitoring line.

18. A blood access system of claim 17, wherein the inside diameter of the lumen of the blood draw member is at least 0.002 inch less than an inside diameter of a lumen of the monitoring line.

19. A blood access system of claim 17, wherein the inside diameter of the lumen of the blood draw member is at least 25% less than an inside diameter of a lumen of the monitoring line.

20. A blood access system of claim 17, wherein the inside diameter of the lumen of the blood draw member is 0.010 inch and an inside diameter of a lumen of the monitoring line is 0.012 inch.

21. A blood access system of claim 1, further comprising an adapter having a distal end configured for attachment to the blood draw member and a proximal end configured for attachment to a sensor casing.

22. A blood access system of claim 21, wherein the adapter defines a lumen having a proximal end with an inside diameter matched to a lumen of the sensor casing and a distal end with an inside diameter matched to the lumen of the blood draw member.

23. A blood access system of claim 22, wherein the adapter includes a surface defining the lumen that is smooth, continuous and void-free so as to avoid clotting during flush and draw pressures of up to 10 psi.

24. A blood access system of claim 23, wherein the inside diameter of the proximal end of the lumen of the adapter is 0.020 inch and the inside diameter of the distal end of the lumen of the adapter is 0.010 inch.

25. A blood access system of claim 23, further comprising the sensor casing.

26. A blood access system of claim 25, wherein the sensor casing is configured to support a blood parameter sensor, the sensor casing including a proximal end configured for attachment to a monitoring line and a distal end configured for attachment to the proximal end of the adapter.

27. A blood access system of claim 26, further comprising the monitoring line.

28. A blood access system of claim 27, wherein the inside diameter of the lumen of the blood draw member is at least 0.002 inch less than an inside diameter of a lumen of the monitoring line.

29. A blood access system of claim 27, wherein the inside diameter of the lumen of the blood draw member is at least 25% less than an inside diameter of a lumen of the monitoring line.

30. A blood access system of claim 27, wherein the inside diameter of the lumen of the blood draw member is 0.010 inch and an inside diameter of a lumen of the monitoring line is 0.012 inch.

31. A blood access system of claim 27, wherein monitoring line is at least 8 feet long.

32. A blood access system of claim 1, further comprising a guide wire configured to extend through the lumen of the blood draw member.

33. A blood access system of claim 32, further comprising an introducer configured to fit over the guide wire and to be withdrawn from the guide wire after insertion into the patient's vasculature.

34. A blood access system of claim 1, further comprising a tear-away introducer configured to insert into the patient's vasculature, the tear-away introducer defining a lumen configured to receive the blood draw member.

35. A sensor system for sensing a parameter in blood accessed by an access device, the sensor system comprising:
a blood parameter sensor coupled to the access device;
a flow control system configured to draw blood through the access device to the blood parameter sensor and flush the sensor with a calibrant to evacuate the blood; and
a monitor connected in communication with the blood parameter sensor, the monitor configured to receive a blood signal when the sensor is in blood and then to receive a calibration signal when the sensor is in the calibrant and use the calibration signal to determine the parameter from the blood signal.

36. A blood parameter sensor system of claim 35, wherein a first delay between the receipt of the blood signal and the calibration signal is shorter than a second delay between receipt of a previous calibration signal and the blood signal.

37. A blood parameter sensor system of claim 36, wherein the first delay is at least 1.5 minutes shorter than the second delay.

38. A blood parameter sensor system of claim 37, wherein the second delay is at least 2.5 minutes.

39. A blood parameter sensor system of claim 36, wherein the blood signal is received at an end of the blood draw.

40. A blood parameter sensor system of claim 39, wherein the blood draw cycle is longer than the calibrant flush cycle.

41. A blood parameter sensor system for sensing a parameter in blood, the blood parameter sensor system comprising:
a blood sensor;
a flow control system configured to expose the blood parameter sensor to a calibrant; and
a monitor connected in communication with the blood parameter sensor, the monitor configured to receive a calibration signal and a blood signal, wherein a delay occurs between the calibration signal and blood signal,
wherein said monitor is configured to modify the calibration signal to account for the delay and use the modified calibration signal to determine the parameter from the blood signal.

42. A blood parameter sensor system of claim 41, wherein the monitor is configured to receive a second calibration signal and use the second calibration signal to modify the calibration signal.

43. A blood parameter sensor system of claim 42, wherein the monitor is configured to use a statistical method to modify the calibration signal.

44. A blood parameter sensor system of claim 43, wherein the statistical method is an interpolative method.

45. A blood parameter sensor system of claim 44, wherein the interpolative method is a linear or non-linear regression or a logarithmic exponential decay.

46. A blood parameter sensor system for sensing a parameter in blood, the blood parameter sensor system comprising:
a blood parameter sensor;
a flow control system configured to flow blood past the blood parameter sensor; and
a monitor connected in communication with the blood parameter sensor, the monitor configured to receive a blood signal from the blood parameter sensor and determine a blood parameter from the blood signal;
wherein the flow control system is configured to continuously flow blood past the sensor while the blood parameter sensor generates the blood signal.

47. A blood parameter sensor system of claim 46, wherein the blood signal includes a waveform and wherein the flow control system is configured to determine a plateau threshold from the waveform.

48. A blood parameter sensor system of claim 47, wherein the monitor is configured to report blood parameters associated with a portion of the blood signal exceeding the plateau threshold.

49. A blood parameter sensor system of claim 48, wherein the monitor is further configured to use the plateau threshold to determine whether the blood is homogenous.

50. A blood parameter sensor system of claim 48, wherein the monitor is further configured to determine when the waveform does not meet the plateau threshold.

51. A blood parameter sensor system of claim 50, wherein the monitor is further configured to determine when the waveform fails to meet the plateau threshold within a maximum delay period.

52. A blood parameter sensor system of claim 47, wherein the waveform includes a plurality of blood signal readings.

53. A blood parameter sensor system of claim 52, wherein the flow control system is further configured to continuously flow blood until a clinical event occurs.

54. A blood parameter sensor system of claim 53, wherein the clinically event includes a clinically relevant volume of blood.

55. A blood parameter sensor system of claim 52, wherein the flow control system is configured to continuously flow the blood past the sensor and to flush blood out of an access device.

56. A blood parameter sensor system for sensing a parameter in blood accessed by an access device, the blood parameter sensor system comprising:
a blood parameter sensor supported by the access device;
a monitor connected in communication with the blood parameter sensor, the monitor configured to receive a blood signal from the blood parameter sensor and determine a blood parameter waveform from the blood signal and determine a threshold from the blood parameter waveform; and
a flow control system connected in communication with the monitor, configured to draw blood into the access device to the sensor and receive the threshold from the monitor and determine a blood draw volume from the threshold.

57. A blood parameter sensor system of claim 56, wherein the blood draw volume is the volume of blood drawn past the sensor when the threshold is reached by the blood parameter waveform.

58. A blood parameter sensor system of claim 57, wherein the threshold is a plateau threshold.

59. A blood parameter sensor system of claim 58, wherein the flow control system is configured to adapt a flow profile using the blood volume.

60. A blood parameter sensor system of claim 59, wherein the flow profile is adapted by increasing a draw rate over a fixed draw period to reach the blood draw volume.

61. A blood parameter sensor system of claim 59, wherein the flow profile is adapted by modifying a draw period while maintaining a fixed draw rate to reach the blood draw volume.

62. A blood parameter sensor system of claim 59, wherein the flow control system is configured to determine a plurality of blood draw volumes and adapt the flow profile using the plurality of blood draw volumes.

63. A blood parameter sensor system of claim 62, wherein each of the plurality of blood draw volumes is associated with an initialization flow profile.

64. A blood parameter sensor system of claim 63, wherein at least two of the initialization flow profiles have different flow rates.

65. A blood parameter sensor system of claim 59, wherein the flow profile is adapted using a lookup table.
